SEQUENCE LISTING (14 sequences)

SEQ ID NO: 1 - DNA, 2115 bp, Homo sapiens
agtttcccttccgctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggaaaa

SEQ ID NO: 2 - DNA, 2033 bp, Homo sapiens
cttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttaaaaaaaaaaaaaaaaaaaaaaaaaaaaaaaaaaaaaaaaaaaaaaaaaaaaaaaaaa

SEQ ID NO: 3 - DNA, 1995 bp, Homo sapiens
acaggggtgaaggcccagagaccagcagaacggcatcccagccacgacggccactttgctctgtctgctctccgccacggccctgctctgttccctgggacacccccgcccccacctcctcaggctgcctgatctgcccagctttccagctttcctctggattccggcctctggtcatccctccccaccctctctccaaggccctctcctggtctcccttcttctagaaccccttcctccacctccctctctgcagaacttctcctttaccccccaccccccaccactgccccctttccttttctgacctccttttggagggctcagcgctgcccagaccataggagagatgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcatccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctgacctggagctgaggcagccagcagatctcagcagcccagtccaaataaactccctgtcagcagcaaaaa

SEQ ID NO: 4 - DNA, 2448 bp, Homo sapiens
agaacacttacaggatgtgtgtagtgtggcatgacagagaactttggtttcctttaatgtgactgtagacctggcagtgttactataagaatcactggcaatcagacacccgggtgtgctgagctagcactcagtgggggcggctactgctcatgtgattgtggagtagacagttggaagaagtacccagtccatttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactgggacctgcactgaacttaaacaggcatgtcattgcctctgtatttaagccaacagagttacccaacccagagactgttaatcatggatgttagagctcaaacgggcttttatatacactaggaattcttgacgtggggtctctggagctccaggaaattcgggcacatcatatgtccatgaaacttcagataaactagggaaaactgggtgctgaggtgaaagcataacttttttggcacagaaagtctaaaggggccactgattttcaaagagatctgtgatccctttttgttttttgtttttgagatggagtcttgctctgttgcccaggctggagtgcaatggcacaatctcggctcactgcaagctccgcctcctgggttcaagcgattctcctgcctcagcctcctgagtggctgggattacaggcatgcaccaccatgcccagctaatttgttgtatttttagtagagacagggtttcaccatgttggccagtgtggtctcaaactcctgacctcatgatttgcctgcctcggcctcccaaagcactgggattacaggcgtgagccaccacatccagccagtgatccttaaaagattaagagatgactggaccaggtctaccttgatcttgaagattcccttggaatgttgagatttaggcttatttgagcactgcctgcccaactgtcagtgccagtgcatagcccttcttttgtctcccttatgaagactgccctgcagggctgagatgtggcaggagctcccagggaaaaacgaagtgcatttgattggtgtgtattggccaagttttgcttgttgtgtgcttgaaagaaaatatctctgaccaacttctgtattcgtggaccaaactgaagctatatttttcacagaagaagaagcagtgacggggacacaaattctgttgcctggtggaaagaaggcaaaggccttcagcaatctatattaccagcgctggatcctttgacagagagtggtccctaaacttaaatttcaagacggtataggcttgatctgtcttgcttattgttgccccctgcgcctagcacaattctgacacacaattggaacttactaaaaatttttttttactgttaaaaaaaaaaaaaaaaaa

SEQ ID NO: 5 - DNA, 2978 bp, Homo sapiens
cgtcctatctgcagtcggctactttcagtggcagaagaggccacatctgcttcctgtaggccctctgggcagaagcatgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttagcaaccagaggcatcttctggaagatacacttttgtctttgctattatagatgaatatataagcagctgtactctccatcagtgctgcgtgtgtgtgtgtgtgtgtatgtgtgtgtgtgttcagttgagtgaataaatgtcatcctcttctccatcttcatttccttggccttttcgttctattccattttgcattatggcaggcctagggtgagtaacgtggatcttgatcataaatgcaaaattaaaaaatatcttgacctggttttaaatctggcagtttgagcagatcctatgtctctgagagacacattcctcataatggccagcattttgggctacaaggttttgtggttgatgatgaggatggcatgactgcagagccatcctcatctcattttttcacgtcattttcagtaactttcactcattcaaaggcaggttataagtaagtcctggtagcagcctctatggggagatttgagagtgactaaatcttggtatctgccctcaagaacttacagttaaatggggagacaatgttgtcatgaaaaggtattatagtaaggagagaaggagacatacacaggccttcaggaagagacgacagtttggggtgaggtagttggcataggcttatctgtgatgaagtggcctgggagcaccaaggggatgttgaggctagtctgggaggagcaggagttttgtctagggaacttgtaggaaattcttggagctgaaagtcccacaaagaaggccctggcaccaagggagtcagcaaacttcagattttattctctgggcaggcatttcaagtttccttttgctgtgacatactcatccattagacagcctgatacaggcctgtagcctcttccggccgtgtgtgctggggaagccccaggaaacgcacatgcccacacagggagccaagtcgtagcatttgggccttgatctaccttttctgcatcaatacactcttgagcctttgaaaaaagaacgtttcccactaaaaagaaaatgtggatttttaaaatagggactcttcctaggggaaaaaggggggctgggagtgatagagggtttaaaaaataaacaccttcaaactaacttcttcgaacccttttattcactccctgacgactttgtgctggggttggggtaactgaaccgcttatttctgtttaattgcattcaggctggatcttagaagacttttatccttccaccatctctctcagaggaatgagcggggaggttggatttactggtgactgattttctttcatgggccaaggaactgaaagagaatgtgaagcaaggttgtgtcttgcgcatggttaaaaataaagcattgtcctgcttcctaagacttagactggggttgacaattgttttagcaacaagacaattcaactatttctcctaggatttttattattattattttttcacttttctaccaaatgggttacataggaagaatgaactgaaatctgtccagagctccaagtcctttggaagaaagattagatgaacgtaaaaatgttgttgtttgctgtggcagtttacagcatttttcttgcaaaattagtgcaaatctgttggaaatagaacacaattcacaaattggaagtgaactaaaatgtaatgacgaaaagggagtagtgttttgatttggaggaggtgtatattcggcagaggttggactgagagttgggtgttatttaacataattatggtaattgggaaacatttataaacactattgggatggtgataaaatacaaaagggcctatagatgttagaaatgggtcaggttactgaaatgggattcaatttgaaaaaaatttttttaaatagaactcactgaactagattctcctctgagaaccagagaagaccatttcatagttggattcctggagacatgcgctatccaccacgtagccactttccacatgtggccatcaaccacttaagatggggttagtttaaatcaagatgtgctgttataattggtataagcataaaatcacactagattctggagatttaatatgaataataagaatactatttcagtagttttggtatattgtgtgtcaaaaatgataatattttggatgtattgggtgaaataaaatattaacattaaaaaaaaaaa

SEQ ID NO: 6 - DNA, 3216 bp, Homo sapiens
gtctttctgttcactttttttcacaaaatcatccaggctcttcctactctcctctcttaccacctctctcttcttttttttttttttttagttatttcacagatgccactggggtaggtaaactgacccaactctgcagcactcagaagacgaagcaaagccttctacttgagcagtttttccatcactgatatgtgcaggaaatgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggaccgaactcaagactggcaagaaatgtaaaagaagcaccaacagaatatgcatccatatgtgtgaggagttaagtctgtttctgactccaacagggaccattgaatgatcagcatgttgacatcattgtctgggctcaacaggatgtcaaataatatttctcaatttgagaatttttactttagaaatgttcatgttagtgcttgggtcttaagggtccataggataaatgattaaaatttctctcagaaacttatttgggagctttttatattatagccttgaataacaaaatctctccaaaactggttgacatcatgagtagcagaatagtagaacgtttaaacttagctacattttacccaatatacaaactcgatcttgcctttgaagctattggaaagacttgtagggaaaagaggtttgtgttacctgcatcagttcactacacactcttgaaaacaaaatgtcccaatttgactaaccaaccataaatacagtaatgattgtatatttcaagtcagtcttccaaaataagaaatttttgctgtgtcagtctaagaatggtgtttcttaaatgcaaaggagaaatcattttaggcttgatgtaagaaaatgaaaataataaatggtgcaataaaaatatagaatataccaattggatatagggtagatgttccacatacctggcaaacaaatgcttatatctactctgttagattgataagcaaatataggtattaatggagcagtcaacgtatagcacatttatgaggaaagtagagactcactgggtcacatagactaatggataggaatgtgacataatgctgctgaattaatatacttatgggcatctgaatagtttaaaagttagtcagaataggtatcactgggcaagtgaagatagcttaaactgcttcatgcttgacttgatagcaagttaaagtgcaattaatggaatggaggaaaacccagaatatttaattggtctgtaggggtcaatttgctttcattcaccacatctgcatcttgctgttcttcttactaaggaatcagggcaaatcatctgtagtgacatattttagtttgctaatcatttattttaaaatactgaggttgcagccacttaagagtatagcaaaagatggattcagatttttggactttccaaagtacttgagttaaactatttcaaaaatagcctataattttattcaacagtttgaggctattcgaattctcaggtgctgctactgaataatgtaatagtcttcatacaaagtggatagcaaaggttaaaatccatttcaacaaatatgtgagctgagctgctgcacaaaggaatgtgatgtgtgtgtgtgtgtgtgtgtgtgtgtgtgttaggtggggtgggtgacaacagaaatggtgcacgagaaactgatcaaattgacattatattttcagtttgcttatgaagctcaaaatactagagtaaatgggtcattaaagaaaataatatgtgaaattatggagtttagaatacaagtggggtatatatacaaaaagacaaaactgaggttttgtggtggagagattttcttaagtaacactggcattaagttttagctccttagatttgggggtgcaaatattcttttgagtcactgttattttgccaattacacctagaatttcaagcaaccaattcgagataggctgttttagccaggctgcatttgtggacaacttatgtaagaaagacatgttagaatagctgcttgtggtattcttaaaaatagaaacaggaaatatggggaggatacatttagctgtcctcttatcagatgaacacacgaaattgaacagttccttcatgattctctcaaacttaaaagcaaaatatttctgtcttatttaaaatatccttagtatgtcttatagtaaagataatgctgataatgatttcatctctaagatgtattaatatatttgtactgtttgccaaaatcacaaatcatttatgtttttattccttttcaaaatggtgtcagagacatacatgcattttcccaaatgactctacttcactattatttacatggcttatttcattagtttatagagggtttgagaaaaagaatatgtagataatttaatggtttttcacaaattttaagcttgtgattgtgctcaatgagaaggtaaagttattaaaacttatttgaaatcaaa

SEQ ID NO: 7 - DNA, 4774 bp, Homo sapiens
gggggcgggtgcctggagcacggcgctggggccgcccgcagcgctcactcgctcgcactcagtcgcgggaggcttccccgcgccggccgcgtcccgcccgctccccggcaccagaagttcctctgcgcgtccgacggcgacatgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctagcccagctgggggacagtgggctgttgtggctgggtctggggcaggtgcatttgagccagggctggctctgtgagtggcctccttggcctcggccctggttccctccctcctgctctgggctcagatactgtgacatcccagaagcccagcccctcaacccctctggatgctacatggggatgctggacggctcagcccctgttccaaggattttggggtgctgagattctcccctagagacctgaaattcaccagctacagatgccaaatgacttacatcttaagaagtctcagaacgtccagcccttcagcagctctcgttctgagacatgagccttgggatgtggcagcatcagtgggacaagatggacactgggccaccctcccaggcaccagacacagggcacggtggagagacttctcccccgtggccgccttggctcccccgttttgcccgaggctgctcttctgtcagacttcctctttgtaccacagtggctctggggccaggcctgcctgcccactggccatcgccaccttccccagctgcctcctaccagcagtttctctgaagatctgtcaacaggttaagtcaatctggggcttccactgcctgcattccagtccccagagcttggtggtcccgaaacgggaagtacatattggggcatggtggcctccgtgagcaaatggtgtcttgggcaatctgaggccaggacagatgttgccccacccactggagatggtgctgagggaggtgggtggggccttctgggaaggtgagtggagaggggcacctgccccccgccctccccatcccctactcccactgctcagcgcgggccattgcaagggtgccacacaatgtcttgtccaccctgggacacttctgagtatgaagcgggatgctattaaaaactacatggggaaacaggtgcaaaccctggagatggattgtaagagccagtttaaatctgcactctgctgctcctcccccacccccaccttccactccatacaatctgggcctggtggagtcttcgcttcagagccattcggccaggtgcgggtgatgttcccatctcctgcttgtgggcatgccctggctttgtttttatacacataggcaaggtgagtcctctgtggaattgtgattgaaggattttaaagcaggggaggagagtagggggcatctctgtacactctgggggtaaaacagggaaggcagtgcctgagcatggggacaggtgaggtggggctgggcagaccccctgtagcgtttagcaggatgggggccccaggtactgtggagagcatagtccagcctgggcatttgtctcctagcagcctacactggctctgctgagctgggcctgggtgctgaaagccaggatttggggctaggcgggaagatgttcgcccaattgcttggggggttggggggatggaaaaggggagcacctctaggctgcctggcagcagtgagccctgggcctgtggctacagccagggaaccccacctggacacatggccctgcttctaagccccccagttaggcccaaaggaatggtccactgagggcctcctgctctgcctgggctgggccaggggctttgaggagagggtaaacataggcccggagatggggctgacacctcgagtggccagaatatgcccaaaccccggcttctcccttgtccctaggcagaggggggtcccttcttttgttccctctggtcaccacaatgcttgatgccagctgccataggaagagggtgctggctggccatggtggcacacacctgtcctcccagcactttgcagggctgaggtggaaggaccgcttaagcccaggtgttcaaggctgctgtgagctgtgttcgagccactacactccagcctggggacggagcaaaactttgcctcaaaacaaattttaaaaagaaagaaagaaggaaagagggtatgtttttcacaattcatgggggcctgcatggcaggagtggggacaggacacctgctgttcctggagtcgaaggacaagcccacagcccagattccggttctcccaactcaggaagagcatgccctgccctctggggaggctggcctggccccagccctcagctgctgaccttgaggcagagacaacttctaagaatttggctgccagaccccaggcctggctgctgctgtgtggagagggaggcggcccgcagcagaacagccaccgcacttcctcctcagcttcctctggtgcggccctgccctctcttctctggacccttttacaactgaacgcatctgggcttcgtggtttcctgttttcagcgaaatttactctgagctcccagttccatcttcatccatggccacaggccctgcctacaacgcactagggacgtccctccctgctgctgctggggaggggcaggctgctggagccgccctctgagttgcccgggatggtagtgcctctgatgccagccctggtggctgtgggctggggtgcatgggagagctgggtgcgagaacatggcgcctccagggggcgggaggagcactaggggctggggcaggaggctcctggagcgctggattcgtggcacagtctgaggccctgagagggaaatccatgcttttaagaactaattcattgttaggagatcaatcaggaattaggggccatcttacctatctcctgacattcacagtttaatagagacttcctgcctttattccctcccagggagaggctgaaggaatggaattgaaagcaccatttggagggttttgctgacacagcggggactgctcagcactccctaaaaacacaccatggaggccactggtgactgctggtgggcaggctggccctgcctgggggagtccgtggcgatgggcgctggggtggaggtgcaggagccccaggacctgcttttcaaaagacttctgcctgaccagagctcccactacatgcagtggcccagggcagaggggctgatacatggcctttttcagggggtgctcctcgcggggtggacttgggagtgtgcagtgggacagggggctgcaggggtcctgccaccaccgagcaccaacttggcccctggggtcctgcctcatgaatgaggccttccccagggctggcctgactgtgctgggggctgggttaacgttttctcagggaaccacaatgcacgaaagaggaactggggttgctaaccaggatgctgggaacaaaggcctcttgaagcccagccacagcccagctgagcatgaggcccagcccatagacggcacaggccacctggcccattccctgggcattccctgctttgcattgctgcttctcttcaccccatggaggctatgtcaccctaactatcctggaatgtgttgagagggattctgaatgatcaatatagcttggtgagacagtgccgagatagatagccatgtctgccttgggcacgggagagggaagtggcagcatgcatgctgtttcttggccttttctgttagaatacttggtgctttccaacacactttcacatgtgttgtaacttgtttgatccacccccttccctgaaaatcctgggaggttttattgctgccatttaacacagagggcaatagaggttctgaaaggtctgtgtcttgtcaaaacaagtaaacggtggaactacgactaaa

SEQ ID NO: 8 - DNA, 3419 bp, Homo sapiens
ccggcctcagggacgcaccggagccgcctttccgggcctcaggcggattctccggcgcggcccgccccgcccctcggactccccgggccgcccccggcccccattcgggccgggcctcgctgcggcggcgactgagccaggctgggccgcgtccctgagtcccagagtcggcgcggcgcggcaggggcagccttccaccacggggagcccagctgtcagccgcctcacaggaagatgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctgaccatgaggaccagggagctgctacccctccctacagctcctaccctctggctgcaatggggctgcactgtgagccctgcccccaacagatgcatcctgctctgacaggtgggctccttctccaaaggatgcgatacacagaccactgtgcagccttatttctccaatggacatgattcccaagtcatcctgctgccttttttcttatagacacaatgaacagaccacccacaaccttagttctctaagtcatcctgcctgctgccttatttcacagtacatacatttcttagggacacagtacactgaccacatcaccaccctcttcttccagtgctgcgtggaccatctggctgccttttttctccaaaagatgcaatattcagactgactgaccccctgccttatttcaccaaagacacgatgcatagtcaccccggccttgtttctccaatggccgtgatacactagtgatcatgttcagccctgcttccacctgcatagaatcttttcttctcagacagggacagtgcggcctcaacatctcctggagtctagaagctgtttcctttcccctccttcctcctcttgctctagccttaatactggccttttccctccctgccccaagtgaagacagggcactctgcgcccaccacatgcacagctgtgcatggagacctgcaggtgcacgtgctggaacacgtgtggttcccccctggcccagcctcctctgcagtgcccctctcccctgcccatcctccccacggaagcatgtgctggtcacactggttctccaggggtctgtgatggggcccctgggggtcagcttctgtccctctgccttctcacctctttgttcctttcttttcatgtatccattcagttgatgtttattgagcaactacagatgtcagcactgtgttaggtgctgggggccctgcgtgggaagataaagttcctccctcaaggactccccatccagctgggagacagacaactaactacactgcaccctgcggtttgcagggggctcctgcctggctccctgctccacacctcctctgtggctcaaggcttcctggatacctcacccccatcccacccataattcttacccagagcatggggttggggcggaaacctggagagagggacatagcccctcgccacggctagagaatctggtggtgtccaaaatgtctgtccaggtgtgggcaggtgggcaggcaccaaggccctctggacctttcatagcagcagaaaaggcagagcctggggcagggcagggccaggaatgctttggggacaccgaggggactgccccccacccccaccatggtgctattctggggctggggcagtcttttcctggcttgcctctggccagctcctggcctctggtagagtgagacttcagacgttctgatgccttccggatgtcatctctccctgccccaggaatggaagatgtgaggacttctaatttaaatgtgggactcggagggattttgtaaactgggggtatattttggggaaaataaatgtctttgtaaaaagcttaaaaaaaaaaaaaaaaaa

SEQ ID NO: 9 - protein, 451 aa, Homo sapiens
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTRLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLATYAPVISAEKAYHEQLSVADITNACFEPANQMVKCDPGHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 10 - protein, 444 aa, Homo sapiens [sequence omitted]

SEQ ID NO: 11 - DNA, 20 bp, Artificial
  Synthetic primer
cagttccaaaccctggtggt

SEQ ID NO: 12 - DNA, 20 bp, Artificial
  Synthetic primer
ggctcctattgtccctcgtg

SEQ ID NO: 13 - DNA, 20 bp, Artificial
  Synthetic primer
ctcctgttcgacagtcagcc

SEQ ID NO: 14 - DNA, 20 bp, Artificial
  Synthetic primer
ctcctgttcgacagtcagcc

(19) United States
(12) Patent Application Publication
(10) Pub. No.: US 2020/0150109 A1
(43) Pub. Date: May 14, 2020

(54) MICROTUBULE-TARGETING DRUGS AS IMMUNE CHECKPOINT INHIBITORS AND METHODS OF SCREENING NOVEL IMMUNE CHECKPOINT INHIBITORS FOR THE TREATMENT OF CANCERS AND INFECTIOUS DISEASES

(71) Applicants: INSERM (Institut National de la Santé et de la Recherche Médicale), Paris (FR); Université Paul Sabatier Toulouse III, Toulouse (FR); Centre National de la Recherche Scientifique (CNRS), Paris (FR)

(72) Inventors: Jean-Jacques FOURNIE, Toulouse Cedex 1 (FR); Stefania MILLEVOI, Toulouse Cedex 1 (FR); Don-Marc FRANCHINI, Toulouse Cedex 1 (FR); Olivia LANVIN, Toulouse Cedex 1 (FR)

(21) Appl. No.: 16/610,572

(22) PCT Filed: May 4, 2018
(86) PCT No.: PCT/EP2018/061499

§ 371 Date: Nov. 4, 2019

(30) Foreign Application Priority Data
May 5, 2017 (EP) 17305514.6

(51) Int. Cl. G01N 33/50

(52) U.S. Cl. CPC G01N 33/5011; G01N 2500/02

(57) ABSTRACT

The invention is based on the discovery that mRNA encoding various checkpoint proteins such as PD-1 must interact with dynamic microtubules to allow surface expression of these proteins. Any inhibitor of this interaction is therefore a putative immune checkpoint inhibitor that could be suitable for the treatment of cancers and infectious diseases. Accordingly, the present invention relates to using microtubule-targeting drugs as immune checkpoint inhibitor, and a method of screening an immune checkpoint inhibitor comprising a) determining the ability of a test compound to inhibit the interaction of an mRNA sequence encoding for an immune checkpoint protein to a polymerized-tubulin moiety and b) positively selecting the test compound that inhibits said binding.

### FIELD OF THE INVENTION

The present invention relates to use of microtubule-targeting drugs as immune checkpoint inhibitors as well as methods for screening novel immune checkpoint inhibitors for the treatment of cancers and infectious diseases.

### BACKGROUND OF THE INVENTION

The ability of the immune system to detect and eliminate cancer was first proposed over 100 years ago. Since then, T cells reactive against tumor-associated antigens have been detected in the blood of patients with many different types of cancers, suggesting a role for the immune system in fighting cancer. Innate and adaptive immunity maintains effector cells such as lymphocytes and natural killer cells that distinguish normal cells from “modified” cells as in the case of tumor cells. However, most often tumor cells are able to evade immune recognition and destruction. The mechanisms of tumor escape are numerous, but the immunosuppressive action of coinhibitory molecules has emerged this last decade as the most attractive one for imaging new treatments of cancer. Activation of lymphocytes is indeed regulated by both costimulatory and coinhibitory molecules, some of which belong to the IgSF Immunoglobulin superfamily the B7/CD28 superfamily, the C-type lectin-like receptor superfamily and the TNF/TNFR superfamily. The balance between these signals determines the lymphocyte activation and consequently regulates the immune response. These costimulatory and coinhibitory molecules were called “immune checkpoints”. Examples of immune checkpoints include B7H3, B7H4, B7H5/VISTA, BTLA, CTLA-4, KIR2DL1-5, KIR3DL1-3, PD-1, PD-L1, PD-L2, CD277, TIM3, LAG3, and TIGIT. Accordingly, the term “immune checkpoint inhibitor” refers to any compound inhibiting the function of an immune checkpoint and typically include peptides, nucleic acid molecules and small molecules, but currently preferred immune checkpoint inhibitors are antibodies. The immune checkpoint inhibitor is administered for enhancing the proliferation, migration, persistence and/or cytotoxic activity of T and NK cells in a subject and in particular the tumor-infiltrating lymphocytes (TIL). One of the most extensively studied immune checkpoint is programmed cell death protein 1 (PD-1) (also known as CD279), which is an Ig-superfamily type cell surface receptor expressed by activated T lymphocytes, NK, B cells and macrophages. Its structure comprises an extracellular IgV domain, a transmembrane region and an intracellular tail containing two immunoreceptor tyrosine-based inhibitory motifs (ITIMs). PD-1 is the receptor for PD-L1 expressed by most cell types and PD-L2, so called butyrophilin B7-DC, expressed by various types of myeloid cells. PD-1 engagement by its ligands recruits the intracellular phosphatase Shp2 to dephosphorylate CD28 co-stimulatory molecule, and thus inhibit the activation pathway. This interaction controls autoimmunity, but since PD-L1 or PD-L2 expressions also allow cancer immune evasion, monoclonal antibodies targeting this immunosuppressive receptor preserve the antitumor activity of cytolytic lymphocytes. Hence, the anti-PD-1 nivolumab and pembrolizumab have achieved impressive clinical responses in a sizeable fraction of patients afflicted with solid cancers such as melanoma, non-small-cell lung cancer, or renal-cell carcinoma. Resting T cells do not express PD-1 however, and how activation drives PD-1 expression at the T cell surface remains unknown.

### SUMMARY OF THE INVENTION

The present invention relates to use of microtubule-targeting drugs as immune checkpoint inhibitors as well as methods for screening novel immune checkpoint inhibitors for the treatment of cancers and infectious diseases. In particular, the present invention is defined by the claims.

### DETAILED DESCRIPTION OF THE INVENTION

Methods of Screening:

The first object of the present invention relates to a method of screening an immune checkpoint inhibitor comprising a) determining the ability of a test compound to inhibit the binding of an mRNA sequence encoding for an immune checkpoint protein to a polymerized-tubulin moiety and b) positively selecting the test compound that inhibits said binding.

As used herein the term “immune checkpoint protein” has its general meaning in the art and refers to a molecule that is expressed by T cells and NK cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include B7-H3, B7-H4, BTLA, CTLA-4, CD277, KIR, PD-1, LAG-3, TIM-3, TIGIT and VISTA. B7-H3, also called CD276, was originally understood to be a co-stimulatory molecule but is now regarded as co-inhibitory. B7-H4, also called VTCN1, is expressed by tumor cells and tumor-associated macrophages and plays a role in tumor escape. B and T Lymphocyte Attenuator (BTLA), also called CD272, is a ligand of HVEM (Herpesvirus Entry Mediator). Cell surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152 is overexpressed on Treg cells serves to control T cell proliferation. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, short for V-domain Ig suppressor of T cell activation, is primarily expressed on hematopoietic cells so that consistent expression of VISTA on leukocytes within tumors may allow VISTA blockade to be effective across a broad range of solid tumors. As used herein, the term “PD-1” has its general meaning in the art and refers to programmed cell death protein 1 (also known as CD279). PD-1 acts as an immune checkpoint, which upon binding of one of its ligands, PD-L1 or PD-L2, enables Shp2 to dephosphorylate CD28 and inhibits the activation of T cells.

As used herein, the term “immune checkpoint inhibitor” has its general meaning in the art and refers to any compound inhibiting the function of an immune inhibitory checkpoint protein. Inhibition includes reduction of function and full blockade. In particular, the immune checkpoint inhibitor particularly suitable for enhancing the proliferation, migration, persistence and/or cytotoxic activity of CD8+ T cells in the patient and in particular the tumor-infiltrating of CD8+ T cells of the patient. As used herein, the term “CD8+ T cell” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions. As used herein, the term “tumor infiltrating CD8+ T cell” refers to the pool of CD8+ T cells of the patient that have left the blood stream and have migrated into a tumor.

As used herein, the term “mRNA” or “messenger RNA” has its general meaning in the art and refers to a single stranded RNA molecule that is synthesized during transcription, is complementary to one of the strands of double-stranded DNA, and serves to transmit the genetic information contained in DNA to the ribosomes for protein synthesis. The mRNA may be spliced, partially spliced or unspliced. The mRNA sequences encompass the following regions: 5′untranslated region (5′UTR), the open reading frame (ORF), and the 3′untranslated region (3′UTR). In some embodiments, the mRNA sequence of the present invention corresponds to the open reading frame (ORF) sequence. The ORF sequences encoding for immune checkpoint proteins are well known in the art. In some embodiments, wherein the mRNA sequence corresponds to the transcription of a sequence selected from the group consisting of SEQ ID NO:1-8.

>NM_005018.2 Homo sapiens programmed cell death 1 (PDCD1),
mRNA
SEQ ID NO: 1
AGTTTCCCTTCCGCTCACCTCCGCCTGAGCAGTGGAGAAGGCGGCACTCTGGTGGGGCTGCTCCAGGCAT
GCAGATCCCACAGGCGCCCTGGCCAGTCGTCTGGGCGGTGCTACAACTGGGCTGGCGGCCAGGATGGTTC
TTAGACTCCCCAGACAGGCCCTGGAACCCCCCCACCTTCTCCCCAGCCCTGCTCGTGGTGACCGAAGGGG
ACAACGCCACCTTCACCTGCAGCTTCTCCAACACATCGGAGAGCTTCGTGCTAAACTGGTACCGCATGAG
CCCCAGCAACCAGACGGACAAGCTGGCCGCCTTCCCCGAGGACCGCAGCCAGCCCGGCCAGGACTGCCGC
TTCCGTGTCACACAACTGCCCAACGGGCGTGACTTCCACATGAGCGTGGTCAGGGCCCGGCGCAATGACA
GCGGCACCTACCTCTGTGGGGCCATCTCCCTGGCCCCCAAGGCGCAGATCAAAGAGAGCCTGCGGGCAGA
GCTCAGGGTGACAGAGAGAAGGGCAGAAGTGCCCACAGCCCACCCCAGCCCCTCACCCAGGCCAGCCGGC
CAGTTCCAAACCCTGGTGGTTGGTGTCGTGGGCGGCCTGCTGGGCAGCCTGGTGCTGCTAGTCTGGGTCC
TGGCCGTCATCTGCTCCCGGGCCGCACGAGGGACAATAGGAGCCAGGCGCACCGGCCAGCCCCTGAAGGA
GGACCCCTCAGCCGTGCCTGTGTTCTCTGTGGACTATGGGGAGCTGGATTTCCAGTGGCGAGAGAAGACC
CCGGAGCCCCCCGTGCCCTGTGTCCCTGAGCAGACGGAGTATGCCACCATTGTCTTTCCTAGCGGAATGG
GCACCTCATCCCCCGCCCGCAGGGGCTCAGCTGACGGCCCTCGGAGTGCCCAGCCACTGAGGCCTGAGGA
TGGACACTGCTCTTGGCCCCTCTGACCGGCTTCCTTGGCCACCAGTGTTCTGCAGACCCTCCACCATGAG
CCCGGGTCAGCGCATTTCCTCAGGAGAAGCAGGCAGGGTGCAGGCCATTGCAGGCCGTCCAGGGGCTGAG
CTGCCTGGGGGCGACCGGGGCTCCAGCCTGCACCTGCACCAGGCACAGCCCCACCACAGGACTCATGTCT
CAATGCCCACAGTGAGCCCAGGCAGCAGGTGTCACCGTCCCCTACAGGGAGGGCCAGATGCAGTCACTGC
TTCAGGTCCTGCCAGCACAGAGCTGCCTGCGTCCAGCTCCCTGAATCTCTGCTGCTGCTGCTGCTGCTGC
TGCTGCTGCCTGCGGCCCGGGGCTGAAGGCGCCGTGGCCCTGCCTGACGCCCCGGAGCCTCCTGCCTGAA
CTTGGGGGCTGGTTGGAGATGGCCTTGGAGCAGCCAAGGTGCCCCTGGCAGTGGCATCCCGAAACGCCCT
GGACGCAGGGCCCAAGACTGGGCACAGGAGTGGGAGGTACATGGGGCTGGGGACTCCCCAGGAGTTATCT
GCTCCCTGCAGGCCTAGAGAAGTTTCAGGGAAGGTCAGAAGAGCTCCTGGCTGTGGTGGGCAGGGCAGGA
AACCCCTCCACCTTTACACATGCCCAGGCAGCACCTCAGGCCCTTTGTGGGGCAGGGAAGCTGAGGCAGT
AAGCGGGCAGGCAGAGCTGGAGGCCTTTCAGGCCCAGCCAGCACTCTGGCCTCCTGCCGCCGCATTCCAC
CCCAGCCCCTCACACCACTCGGGAGAGGGACATCCTACGGTCCCAAGGTCAGGAGGGCAGGGCTGGGGTT
GACTCAGGCCCCTCCCAGCTGTGGCCACCTGGGTGTTGGGAGGGCAGAAGTGCAGGCACCTAGGGCCCCC
CATGTGCCCACCCTGGGAGCTCTCCTTGGAACCCATTCCTGAAATTATTTAAAGGGGTTGGCCGGGCTCC
CACCAGGGCCTGGGTGGGAAGGTACAGGCGTTCCCCCGGGGCCTAGTACCCCCGCCGTGGCCTATCCACT
CCTCACATCCACACACTGCACCCCCACTCCTGGGGCAGGGCCACCAGCATCCAGGCGGCCAGCAGGCACC
TGAGTGGCTGGGACAAGGGATCCCCCTTCCCTGTGGTTCTATTATATTATAATTATAATTAAATATGAGA
GCATGCTAAGGAAAA
>NM_005214.4 Homo sapiens cytotoxic T-lymphocyte associated
protein 4 (CTLA4), transcript variant 1, mRNA
SEQ ID NO: 2
CTTCTGTGTGTGCACATGTGTAATACATATCTGGGATCAAAGCTATCTATATAAAGTCCTTGATTCTGTG
TGGGTTCAAACACATTTCAAAGCTTCAGGATCCTGAAAGGTTTTGCTCTACTTCCTGAAGACCTGAACAC
CGCTCCCATAAAGCCATGGCTTGCCTTGGATTTCAGCGGCACAAGGCTCAGCTGAACCTGGCTACCAGGA
CCTGGCCCTGCACTCTCCTGTTTTTTCTTCTCTTCATCCCTGTCTTCTGCAAAGCAATGCACGTGGCCCA
GCCTGCTGTGGTACTGGCCAGCAGCCGAGGCATCGCCAGCTTTGTGTGTGAGTATGCATCTCCAGGCAAA
GCCACTGAGGTCCGGGTGACAGTGCTTCGGCAGGCTGACAGCCAGGTGACTGAAGTCTGTGCGGCAACCT
ACATGATGGGGAATGAGTTGACCTTCCTAGATGATTCCATCTGCACGGGCACCTCCAGTGGAAATCAAGT
GAACCTCACTATCCAAGGACTGAGGGCCATGGACACGGGACTCTACATCTGCAAGGTGGAGCTCATGTAC
CCACCGCCATACTACCTGGGCATAGGCAACGGAACCCAGATTTATGTAATTGATCCAGAACCGTGCCCAG
ATTCTGACTTCCTCCTCTGGATCCTTGCAGCAGTTAGTTCGGGGTTGTTTTTTTATAGCTTTCTCCTCAC
AGCTGTTTCTTTGAGCAAAATGCTAAAGAAAAGAAGCCCTCTTACAACAGGGGTCTATGTGAAAATGCCC
CCAACAGAGCCAGAATGTGAAAAGCAATTTCAGCCTTATTTTATTCCCATCAATTGAGAAACCATTATGA
AGAAGAGAGTCCATATTTCAATTTCCAAGAGCTGAGGCAATTCTAACTTTTTTGCTATCCAGCTATTTTT
ATTTGTTTGTGCATTTGGGGGGAATTCATCTCTCTTTAATATAAAGTTGGATGCGGAACCCAAATTACGT
GTACTACAATTTAAAGCAAAGGAGTAGAAAGACAGAGCTGGGATGTTTCTGTCACATCAGCTCCACTTTC
AGTGAAAGCATCACTTGGGATTAATATGGGGATGCAGCATTATGATGTGGGTCAAGGAATTAAGTTAGGG
AATGGCACAGCCCAAAGAAGGAAAAGGCAGGGAGCGAGGGAGAAGACTATATTGTACACACCTTATATTT
ACGTATGAGACGTTTATAGCCGAAATGATCTTTTCAAGTTAAATTTTATGCCTTTTATTTCTTAAACAAA
TGTATGATTACATCAAGGCTTCAAAAATACTCACATGGCTATGTTTTAGCCAGTGATGCTAAAGGTTGTA
TTGCATATATACATATATATATATATATATATATATATATATATATATATATATATATATATATATATTT
TAATTTGATAGTATTGTGCATAGAGCCACGTATGTTTTTGTGTATTTGTTAATGGTTTGAATATAAACAC
TATATGGCAGTGTCTTTCCACCTTGGGTCCCAGGGAAGTTTTGTGGAGGAGCTCAGGACACTAATACACC
AGGTAGAACACAAGGTCATTTGCTAACTAGCTTGGAAACTGGATGAGGTCATAGCAGTGCTTGATTGCGT
GGAATTGTGCTGAGTTGGTGTTGACATGTGCTTTGGGGCTTTTACACCAGTTCCTTTCAATGGTTTGCAA
GGAAGCCACAGCTGGTGGTATCTGAGTTGACTTGACAGAACACTGTCTTGAAGACAATGGCTTACTCCAG
GAGACCCACAGGTATGACCTTCTAGGAAGCTCCAGTTCGATGGGCCCAATTCTTACAAACATGTGGTTAA
TGCCATGGACAGAAGAAGGCAGCAGGTGGCAGAATGGGGTGCATGAAGGTTTCTGAAAATTAACACTGCT
TGTGTTTTTAACTCAATATTTTCCATGAAAATGCAACAACATGTATAATATTTTTAATTAAATAAAAATC
TGTGGTGGTCGTTTTAAAAAAAAAAAAAAAAAAAAAAAAAAAAAAAAAAAAAAAAAAAAAAAAAAAAAAA
AAA
>NM_002286.5 Homo sapiens lymphocyte activating 3 (LAG3), mRNA
SEQ ID NO: 3
ACAGGGGTGAAGGCCCAGAGACCAGCAGAACGGCATCCCAGCCACGACGGCCACTTTGCTCTGTCTGCTC
TCCGCCACGGCCCTGCTCTGTTCCCTGGGACACCCCCGCCCCCACCTCCTCAGGCTGCCTGATCTGCCCA
GCTTTCCAGCTTTCCTCTGGATTCCGGCCTCTGGTCATCCCTCCCCACCCTCTCTCCAAGGCCCTCTCCT
GGTCTCCCTTCTTCTAGAACCCCTTCCTCCACCTCCCTCTCTGCAGAACTTCTCCTTTACCCCCCACCCC
CCACCACTGCCCCCTTTCCTTTTCTGACCTCCTTTTGGAGGGCTCAGCGCTGCCCAGACCATAGGAGAGA
TGTGGGAGGCTCAGTTCCTGGGCTTGCTGTTTCTGCAGCCGCTTTGGGTGGCTCCAGTGAAGCCTCTCCA
GCCAGGGGCTGAGGTCCCGGTGGTGTGGGCCCAGGAGGGGGCTCCTGCCCAGCTCCCCTGCAGCCCCACA
ATCCCCCTCCAGGATCTCAGCCTTCTGCGAAGAGCAGGGGTCACTTGGCAGCATCAGCCAGACAGTGGCC
CGCCCGCTGCCGCCCCCGGCCATCCCCTGGCCCCCGGCCCTCACCCGGCGGCGCCCTCCTCCTGGGGGCC
CAGGCCCCGCCGCTACACGGTGCTGAGCGTGGGTCCCGGAGGCCTGCGCAGCGGGAGGCTGCCCCTGCAG
CCCCGCGTCCAGCTGGATGAGCGCGGCCGGCAGCGCGGGGACTTCTCGCTATGGCTGCGCCCAGCCCGGC
GCGCGGACGCCGGCGAGTACCGCGCCGCGGTGCACCTCAGGGACCGCGCCCTCTCCTGCCGCCTCCGTCT
GCGCCTGGGCCAGGCCTCGATGACTGCCAGCCCCCCAGGATCTCTCAGAGCCTCCGACTGGGTCATTTTG
AACTGCTCCTTCAGCCGCCCTGACCGCCCAGCCTCTGTGCATTGGTTCCGGAACCGGGGCCAGGGCCGAG
TCCCTGTCCGGGAGTCCCCCCATCACCACTTAGCGGAAAGCTTCCTCTTCCTGCCCCAAGTCAGCCCCAT
GGACTCTGGGCCCTGGGGCTGCATCCTCACCTACAGAGATGGCTTCAACGTCTCCATCATGTATAACCTC
ACTGTTCTGGGTCTGGAGCCCCCAACTCCCTTGACAGTGTACGCTGGAGCAGGTTCCAGGGTGGGGCTGC
CCTGCCGCCTGCCTGCTGGTGTGGGGACCCGGTCTTTCCTCACTGCCAAGTGGACTCCTCCTGGGGGAGG
CCCTGACCTCCTGGTGACTGGAGACAATGGCGACTTTACCCTTCGACTAGAGGATGTGAGCCAGGCCCAG
GCTGGGACCTACACCTGCCATATCCATCTGCAGGAACAGCAGCTCAATGCCACTGTCACATTGGCAATCA
TCACAGTGACTCCCAAATCCTTTGGGTCACCTGGATCCCTGGGGAAGCTGCTTTGTGAGGTGACTCCAGT
ATCTGGACAAGAACGCTTTGTGTGGAGCTCTCTGGACACCCCATCCCAGAGGAGTTTCTCAGGACCTTGG
CTGGAGGCACAGGAGGCCCAGCTCCTTTCCCAGCCTTGGCAATGCCAGCTGTACCAGGGGGAGAGGCTTC
TTGGAGCAGCAGTGTACTTCACAGAGCTGTCTAGCCCAGGTGCCCAACGCTCTGGGAGAGCCCCAGGTGC
CCTCCCAGCAGGCCACCTCCTGCTGTTTCTCATCCTTGGTGTCCTTTCTCTGCTCCTTTTGGTGACTGGA
GCCTTTGGCTTTCACCTTTGGAGAAGACAGTGGCGACCAAGACGATTTTCTGCCTTAGAGCAAGGGATTC
ACCCTCCGCAGGCTCAGAGCAAGATAGAGGAGCTGGAGCAAGAACCGGAGCCGGAGCCGGAGCCGGAACC
GGAGCCCGAGCCCGAGCCCGAGCCGGAGCAGCTCTGACCTGGAGCTGAGGCAGCCAGCAGATCTCAGCAG
CCCAGTCCAAATAAACTCCCTGTCAGCAGCAAAAA
>NM_032782.4:255-1160 Homo sapiens T-Cell Immunoglobulin And
Mucin Domain-Containing Protein 3 (TIM3), mRNA
SEQ ID NO: 4
AGAACACTTACAGGATGTGTGTAGTGTGGCATGACAGAGAACTTTGGTTTCCTTTAATGTGACTGTAGAC
CTGGCAGTGTTACTATAAGAATCACTGGCAATCAGACACCCGGGTGTGCTGAGCTAGCACTCAGTGGGGG
CGGCTACTGCTCATGTGATTGTGGAGTAGACAGTTGGAAGAAGTACCCAGTCCATTTGGAGAGTTAAAAC
TGTGCCTAACAGAGGTGTCCTCTGACTTTTCTTCTGCAAGCTCCATGTTTTCACATCTTCCCTTTGACTG
TGTCCTGCTGCTGCTGCTGCTACTACTTACAAGGTCCTCAGAAGTGGAATACAGAGCGGAGGTCGGTCAG
AATGCCTATCTGCCCTGCTTCTACACCCCAGCCGCCCCAGGGAACCTCGTGCCCGTCTGCTGGGGCAAAG
GAGCCTGTCCTGTGTTTGAATGTGGCAACGTGGTGCTCAGGACTGATGAAAGGGATGTGAATTATTGGAC
ATCCAGATACTGGCTAAATGGGGATTTCCGCAAAGGAGATGTGTCCCTGACCATAGAGAATGTGACTCTA
GCAGACAGTGGGATCTACTGCTGCCGGATCCAAATCCCAGGCATAATGAATGATGAAAAATTTAACCTGA
AGTTGGTCATCAAACCAGCCAAGGTCACCCCTGCACCGACTCGGCAGAGAGACTTCACTGCAGCCTTTCC
AAGGATGCTTACCACCAGGGGACATGGCCCAGCAGAGACACAGACACTGGGGAGCCTCCCTGATATAAAT
CTAACACAAATATCCACATTGGCCAATGAGTTACGGGACTCTAGATTGGCCAATGACTTACGGGACTCTG
GAGCAACCATCAGAATAGGCATCTACATCGGAGCAGGGATCTGTGCTGGGCTGGCTCTGGCTCTTATCTT
CGGCGCTTTAATTTTCAAATGGTATTCTCATAGCAAAGAGAAGATACAGAATTTAAGCCTCATCTCTTTG
GCCAACCTCCCTCCCTCAGGATTGGCAAATGCAGTAGCAGAGGGAATTCGCTCAGAAGAAAACATCTATA
CCATTGAAGAGAACGTATATGAAGTGGAGGAGCCCAATGAGTATTATTGCTATGTCAGCAGCAGGCAGCA
ACCCTCACAACCTTTGGGTTGTCGCTTTGCAATGCCATAGATCCAACCACCTTATTTTTGAGCTTGGTGT
TTTGTCTTTTTCAGAAACTATGAGCTGTGTCACCTGACTGGTTTTGGAGGTTCTGTCCACTGCTATGGAG
CAGAGTTTTCCCATTTTCAGAAGATAATGACTCACATGGGAATTGAACTGGGACCTGCACTGAACTTAAA
CAGGCATGTCATTGCCTCTGTATTTAAGCCAACAGAGTTACCCAACCCAGAGACTGTTAATCATGGATGT
TAGAGCTCAAACGGGCTTTTATATACACTAGGAATTCTTGACGTGGGGTCTCTGGAGCTCCAGGAAATTC
GGGCACATCATATGTCCATGAAACTTCAGATAAACTAGGGAAAACTGGGTGCTGAGGTGAAAGCATAACT
TTTTTGGCACAGAAAGTCTAAAGGGGCCACTGATTTTCAAAGAGATCTGTGATCCCTTTTTGTTTTTTGT
TTTTGAGATGGAGTCTTGCTCTGTTGCCCAGGCTGGAGTGCAATGGCACAATCTCGGCTCACTGCAAGCT
CCGCCTCCTGGGTTCAAGCGATTCTCCTGCCTCAGCCTCCTGAGTGGCTGGGATTACAGGCATGCACCAC
CATGCCCAGCTAATTTGTTGTATTTTTAGTAGAGACAGGGTTTCACCATGTTGGCCAGTGTGGTCTCAAA
CTCCTGACCTCATGATTTGCCTGCCTCGGCCTCCCAAAGCACTGGGATTACAGGCGTGAGCCACCACATC
CAGCCAGTGATCCTTAAAAGATTAAGAGATGACTGGACCAGGTCTACCTTGATCTTGAAGATTCCCTTGG
AATGTTGAGATTTAGGCTTATTTGAGCACTGCCTGCCCAACTGTCAGTGCCAGTGCATAGCCCTTCTTTT
GTCTCCCTTATGAAGACTGCCCTGCAGGGCTGAGATGTGGCAGGAGCTCCCAGGGAAAAACGAAGTGCAT
TTGATTGGTGTGTATTGGCCAAGTTTTGCTTGTTGTGTGCTTGAAAGAAAATATCTCTGACCAACTTCTG
TATTCGTGGACCAAACTGAAGCTATATTTTTCACAGAAGAAGAAGCAGTGACGGGGACACAAATTCTGTT
GCCTGGTGGAAAGAAGGCAAAGGCCTTCAGCAATCTATATTACCAGCGCTGGATCCTTTGACAGAGAGTG
GTCCCTAAACTTAAATTTCAAGACGGTATAGGCTTGATCTGTCTTGCTTATTGTTGCCCCCTGCGCCTAG
CACAATTCTGACACACAATTGGAACTTACTAATTTTTTTTTACTGTTAAAAAAAAAAAAAAAAAAAAA
>NM_173799.3 Homo sapiens T-cell immunoreceptor with Ig and ITIM
domains (TIGIT), mRNA
SEQ ID NO: 5
CGTCCTATCTGCAGTCGGCTACTTTCAGTGGCAGAAGAGGCCACATCTGCTTCCTGTAGGCCCTCTGGGC
AGAAGCATGCGCTGGTGTCTCCTCCTGATCTGGGCCCAGGGGCTGAGGCAGGCTCCCCTCGCCTCAGGAA
TGATGACAGGCACAATAGAAACAACGGGGAACATTTCTGCAGAGAAAGGTGGCTCTATCATCTTACAATG
TCACCTCTCCTCCACCACGGCACAAGTGACCCAGGTCAACTGGGAGCAGCAGGACCAGCTTCTGGCCATT
TGTAATGCTGACTTGGGGTGGCACATCTCCCCATCCTTCAAGGATCGAGTGGCCCCAGGTCCCGGCCTGG
GCCTCACCCTCCAGTCGCTGACCGTGAACGATACAGGGGAGTACTTCTGCATCTATCACACCTACCCTGA
TGGGACGTACACTGGGAGAATCTTCCTGGAGGTCCTAGAAAGCTCAGTGGCTGAGCACGGTGCCAGGTTC
CAGATTCCATTGCTTGGAGCCATGGCCGCGACGCTGGTGGTCATCTGCACAGCAGTCATCGTGGTGGTCG
CGTTGACTAGAAAGAAGAAAGCCCTCAGAATCCATTCTGTGGAAGGTGACCTCAGGAGAAAATCAGCTGG
ACAGGAGGAATGGAGCCCCAGTGCTCCCTCACCCCCAGGAAGCTGTGTCCAGGCAGAAGCTGCACCTGCT
GGGCTCTGTGGAGAGCAGCGGGGAGAGGACTGTGCCGAGCTGCATGACTACTTCAATGTCCTGAGTTACA
GAAGCCTGGGTAACTGCAGCTTCTTCACAGAGACTGGTTAGCAACCAGAGGCATCTTCTGGAAGATACAC
TTTTGTCTTTGCTATTATAGATGAATATATAAGCAGCTGTACTCTCCATCAGTGCTGCGTGTGTGTGTGT
GTGTGTATGTGTGTGTGTGTTCAGTTGAGTGAATAAATGTCATCCTCTTCTCCATCTTCATTTCCTTGGC
CTTTTCGTTCTATTCCATTTTGCATTATGGCAGGCCTAGGGTGAGTAACGTGGATCTTGATCATAAATGC
AAAATTAAAAAATATCTTGACCTGGTTTTAAATCTGGCAGTTTGAGCAGATCCTATGTCTCTGAGAGACA
CATTCCTCATAATGGCCAGCATTTTGGGCTACAAGGTTTTGTGGTTGATGATGAGGATGGCATGACTGCA
GAGCCATCCTCATCTCATTTTTTCACGTCATTTTCAGTAACTTTCACTCATTCAAAGGCAGGTTATAAGT
AAGTCCTGGTAGCAGCCTCTATGGGGAGATTTGAGAGTGACTAAATCTTGGTATCTGCCCTCAAGAACTT
ACAGTTAAATGGGGAGACAATGTTGTCATGAAAAGGTATTATAGTAAGGAGAGAAGGAGACATACACAGG
CCTTCAGGAAGAGACGACAGTTTGGGGTGAGGTAGTTGGCATAGGCTTATCTGTGATGAAGTGGCCTGGG
AGCACCAAGGGGATGTTGAGGCTAGTCTGGGAGGAGCAGGAGTTTTGTCTAGGGAACTTGTAGGAAATTC
TTGGAGCTGAAAGTCCCACAAAGAAGGCCCTGGCACCAAGGGAGTCAGCAAACTTCAGATTTTATTCTCT
GGGCAGGCATTTCAAGTTTCCTTTTGCTGTGACATACTCATCCATTAGACAGCCTGATACAGGCCTGTAG
CCTCTTCCGGCCGTGTGTGCTGGGGAAGCCCCAGGAAACGCACATGCCCACACAGGGAGCCAAGTCGTAG
CATTTGGGCCTTGATCTACCTTTTCTGCATCAATACACTCTTGAGCCTTTGAAAAAAGAACGTTTCCCAC
TAAAAAGAAAATGTGGATTTTTAAAATAGGGACTCTTCCTAGGGGAAAAAGGGGGGCTGGGAGTGATAGA
GGGTTTAAAAAATAAACACCTTCAAACTAACTTCTTCGAACCCTTTTATTCACTCCCTGACGACTTTGTG
CTGGGGTTGGGGTAACTGAACCGCTTATTTCTGTTTAATTGCATTCAGGCTGGATCTTAGAAGACTTTTA
TCCTTCCACCATCTCTCTCAGAGGAATGAGCGGGGAGGTTGGATTTACTGGTGACTGATTTTCTTTCATG
GGCCAAGGAACTGAAAGAGAATGTGAAGCAAGGTTGTGTCTTGCGCATGGTTAAAAATAAAGCATTGTCC
TGCTTCCTAAGACTTAGACTGGGGTTGACAATTGTTTTAGCAACAAGACAATTCAACTATTTCTCCTAGG
ATTTTTATTATTATTATTTTTTCACTTTTCTACCAAATGGGTTACATAGGAAGAATGAACTGAAATCTGT
CCAGAGCTCCAAGTCCTTTGGAAGAAAGATTAGATGAACGTAAAAATGTTGTTGTTTGCTGTGGCAGTTT
ACAGCATTTTTCTTGCAAAATTAGTGCAAATCTGTTGGAAATAGAACACAATTCACAAATTGGAAGTGAA
CTAAAATGTAATGACGAAAAGGGAGTAGTGTTTTGATTTGGAGGAGGTGTATATTCGGCAGAGGTTGGAC
TGAGAGTTGGGTGTTATTTAACATAATTATGGTAATTGGGAAACATTTATAAACACTATTGGGATGGTGA
TAAAATACAAAAGGGCCTATAGATGTTAGAAATGGGTCAGGTTACTGAAATGGGATTCAATTTGAAAAAA
ATTTTTTTAAATAGAACTCACTGAACTAGATTCTCCTCTGAGAACCAGAGAAGACCATTTCATAGTTGGA
TTCCTGGAGACATGCGCTATCCACCACGTAGCCACTTTCCACATGTGGCCATCAACCACTTAAGATGGGG
TTAGTTTAAATCAAGATGTGCTGTTATAATTGGTATAAGCATAAAATCACACTAGATTCTGGAGATTTAA
TATGAATAATAAGAATACTATTTCAGTAGTTTTGGTATATTGTGTGTCAAAAATGATAATATTTTGGATG
TATTGGGTGAAATAAAATATTAACATTAAAAAAAAAAA
>NM_181780.3 Homo sapiens B and T lymphocyte associated (BTLA),
transcript variant 1, mRNA
SEQ ID NO: 6
GTCTTTCTGTTCACTTTTTTTCACAAAATCATCCAGGCTCTTCCTACTCTCCTCTCTTACCACCTCTCTC
TTCTTTTTTTTTTTTTTTTAGTTATTTCACAGATGCCACTGGGGTAGGTAAACTGACCCAACTCTGCAGC
ACTCAGAAGACGAAGCAAAGCCTTCTACTTGAGCAGTTTTTCCATCACTGATATGTGCAGGAAATGAAGA
CATTGCCTGCCATGCTTGGAACTGGGAAATTATTTTGGGTCTTCTTCTTAATCCCATATCTGGACATCTG
GAACATCCATGGGAAAGAATCATGTGATGTACAGCTTTATATAAAGAGACAATCTGAACACTCCATCTTA
GCAGGAGATCCCTTTGAACTAGAATGCCCTGTGAAATACTGTGCTAACAGGCCTCATGTGACTTGGTGCA
AGCTCAATGGAACAACATGTGTAAAACTTGAAGATAGACAAACAAGTTGGAAGGAAGAGAAGAACATTTC
ATTTTTCATTCTACATTTTGAACCAGTGCTTCCTAATGACAATGGGTCATACCGCTGTTCTGCAAATTTT
CAGTCTAATCTCATTGAAAGCCACTCAACAACTCTTTATGTGACAGATGTAAAAAGTGCCTCAGAACGAC
CCTCCAAGGACGAAATGGCAAGCAGACCCTGGCTCCTGTATAGTTTACTTCCTTTGGGGGGATTGCCTCT
ACTCATCACTACCTGTTTCTGCCTGTTCTGCTGCCTGAGAAGGCACCAAGGAAAGCAAAATGAACTCTCT
GACACAGCAGGAAGGGAAATTAACCTGGTTGATGCTCACCTTAAGAGTGAGCAAACAGAAGCAAGCACCA
GGCAAAATTCCCAAGTACTGCTATCAGAAACTGGAATTTATGATAATGACCCTGACCTTTGTTTCAGGAT
GCAGGAAGGGTCTGAAGTTTATTCTAATCCATGCCTGGAAGAAAACAAACCAGGCATTGTTTATGCTTCC
CTGAACCATTCTGTCATTGGACCGAACTCAAGACTGGCAAGAAATGTAAAAGAAGCACCAACAGAATATG
CATCCATATGTGTGAGGAGTTAAGTCTGTTTCTGACTCCAACAGGGACCATTGAATGATCAGCATGTTGA
CATCATTGTCTGGGCTCAACAGGATGTCAAATAATATTTCTCAATTTGAGAATTTTTACTTTAGAAATGT
TCATGTTAGTGCTTGGGTCTTAAGGGTCCATAGGATAAATGATTAAAATTTCTCTCAGAAACTTATTTGG
GAGCTTTTTATATTATAGCCTTGAATAACAAAATCTCTCCAAAACTGGTTGACATCATGAGTAGCAGAAT
AGTAGAACGTTTAAACTTAGCTACATTTTACCCAATATACAAACTCGATCTTGCCTTTGAAGCTATTGGA
AAGACTTGTAGGGAAAAGAGGTTTGTGTTACCTGCATCAGTTCACTACACACTCTTGAAAACAAAATGTC
CCAATTTGACTAACCAACCATAAATACAGTAATGATTGTATATTTCAAGTCAGTCTTCCAAAATAAGAAA
TTTTTGCTGTGTCAGTCTAAGAATGGTGTTTCTTAAATGCAAAGGAGAAATCATTTTAGGCTTGATGTAA
GAAAATGAAAATAATAAATGGTGCAATAAAAATATAGAATATACCAATTGGATATAGGGTAGATGTTCCA
CATACCTGGCAAACAAATGCTTATATCTACTCTGTTAGATTGATAAGCAAATATAGGTATTAATGGAGCA
GTCAACGTATAGCACATTTATGAGGAAAGTAGAGACTCACTGGGTCACATAGACTAATGGATAGGAATGT
GACATAATGCTGCTGAATTAATATACTTATGGGCATCTGAATAGTTTAAAAGTTAGTCAGAATAGGTATC
ACTGGGCAAGTGAAGATAGCTTAAACTGCTTCATGCTTGACTTGATAGCAAGTTAAAGTGCAATTAATGG
AATGGAGGAAAACCCAGAATATTTAATTGGTCTGTAGGGGTCAATTTGCTTTCATTCACCACATCTGCAT
CTTGCTGTTCTTCTTACTAAGGAATCAGGGCAAATCATCTGTAGTGACATATTTTAGTTTGCTAATCATT
TATTTTAAAATACTGAGGTTGCAGCCACTTAAGAGTATAGCAAAAGATGGATTCAGATTTTTGGACTTTC
CAAAGTACTTGAGTTAAACTATTTCAAAAATAGCCTATAATTTTATTCAACAGTTTGAGGCTATTCGAAT
TCTCAGGTGCTGCTACTGAATAATGTAATAGTCTTCATACAAAGTGGATAGCAAAGGTTAAAATCCATTT
CAACAAATATGTGAGCTGAGCTGCTGCACAAAGGAATGTGATGTGTGTGTGTGTGTGTGTGTGTGTGTGT
GTTAGGTGGGGTGGGTGACAACAGAAATGGTGCACGAGAAACTGATCAAATTGACATTATATTTTCAGTT
TGCTTATGAAGCTCAAAATACTAGAGTAAATGGGTCATTAAAGAAAATAATATGTGAAATTATGGAGTTT
AGAATACAAGTGGGGTATATATACAAAAAGACAAAACTGAGGTTTTGTGGTGGAGAGATTTTCTTAAGTA
ACACTGGCATTAAGTTTTAGCTCCTTAGATTTGGGGGTGCAAATATTCTTTTGAGTCACTGTTATTTTGC
CAATTACACCTAGAATTTCAAGCAACCAATTCGAGATAGGCTGTTTTAGCCAGGCTGCATTTGTGGACAA
CTTATGTAAGAAAGACATGTTAGAATAGCTGCTTGTGGTATTCTTAAAAATAGAAACAGGAAATATGGGG
AGGATACATTTAGCTGTCCTCTTATCAGATGAACACACGAAATTGAACAGTTCCTTCATGATTCTCTCAA
ACTTAAAAGCAAAATATTTCTGTCTTATTTAAAATATCCTTAGTATGTCTTATAGTAAAGATAATGCTGA
TAATGATTTCATCTCTAAGATGTATTAATATATTTGTACTGTTTGCCAAAATCACAAATCATTTATGTTT
TTATTCCTTTTCAAAATGGTGTCAGAGACATACATGCATTTTCCCAAATGACTCTACTTCACTATTATTT
ACATGGCTTATTTCATTAGTTTATAGAGGGTTTGAGAAAAAGAATATGTAGATAATTTAATGGTTTTTCA
CAAATTTTAAGCTTGTGATTGTGCTCAATGAGAAGGTAAAGTTATTAACTTATTTGAAATCAAA
>NM_022153.1 Homo sapiens V-Domain Ig Suppressor Of T Cell
Activation (VISTA), mRNA
SEQ ID NO: 7
GGGGGCGGGTGCCTGGAGCACGGCGCTGGGGCCGCCCGCAGCGCTCACTCGCTCGCACTCAGTCGCGGGA
GGCTTCCCCGCGCCGGCCGCGTCCCGCCCGCTCCCCGGCACCAGAAGTTCCTCTGCGCGTCCGACGGCGA
CATGGGCGTCCCCACGGCCCTGGAGGCCGGCAGCTGGCGCTGGGGATCCCTGCTCTTCGCTCTCTTCCTG
GCTGCGTCCCTAGGTCCGGTGGCAGCCTTCAAGGTCGCCACGCCGTATTCCCTGTATGTCTGTCCCGAGG
GGCAGAACGTCACCCTCACCTGCAGGCTCTTGGGCCCTGTGGACAAAGGGCACGATGTGACCTTCTACAA
GACGTGGTACCGCAGCTCGAGGGGCGAGGTGCAGACCTGCTCAGAGCGCCGGCCCATCCGCAACCTCACG
TTCCAGGACCTTCACCTGCACCATGGAGGCCACCAGGCTGCCAACACCAGCCACGACCTGGCTCAGCGCC
ACGGGCTGGAGTCGGCCTCCGACCACCATGGCAACTTCTCCATCACCATGCGCAACCTGACCCTGCTGGA
TAGCGGCCTCTACTGCTGCCTGGTGGTGGAGATCAGGCACCACCACTCGGAGCACAGGGTCCATGGTGCC
ATGGAGCTGCAGGTGCAGACAGGCAAAGATGCACCATCCAACTGTGTGGTGTACCCATCCTCCTCCCAGG
ATAGTGAAAACATCACGGCTGCAGCCCTGGCTACGGGTGCCTGCATCGTAGGAATCCTCTGCCTCCCCCT
CATCCTGCTCCTGGTCTACAAGCAAAGGCAGGCAGCCTCCAACCGCCGTGCCCAGGAGCTGGTGCGGATG
GACAGCAACATTCAAGGGATTGAAAACCCCGGCTTTGAAGCCTCACCACCTGCCCAGGGGATACCCGAGG
CCAAAGTCAGGCACCCCCTGTCCTATGTGGCCCAGCGGCAGCCTTCTGAGTCTGGGCGGCATCTGCTTTC
GGAGCCCAGCACCCCCCTGTCTCCTCCAGGCCCCGGAGACGTCTTCTTCCCATCCCTGGACCCTGTCCCT
GACTCTCCAAACTTTGAGGTCATCTAGCCCAGCTGGGGGACAGTGGGCTGTTGTGGCTGGGTCTGGGGCA
GGTGCATTTGAGCCAGGGCTGGCTCTGTGAGTGGCCTCCTTGGCCTCGGCCCTGGTTCCCTCCCTCCTGC
TCTGGGCTCAGATACTGTGACATCCCAGAAGCCCAGCCCCTCAACCCCTCTGGATGCTACATGGGGATGC
TGGACGGCTCAGCCCCTGTTCCAAGGATTTTGGGGTGCTGAGATTCTCCCCTAGAGACCTGAAATTCACC
AGCTACAGATGCCAAATGACTTACATCTTAAGAAGTCTCAGAACGTCCAGCCCTTCAGCAGCTCTCGTTC
TGAGACATGAGCCTTGGGATGTGGCAGCATCAGTGGGACAAGATGGACACTGGGCCACCCTCCCAGGCAC
CAGACACAGGGCACGGTGGAGAGACTTCTCCCCCGTGGCCGCCTTGGCTCCCCCGTTTTGCCCGAGGCTG
CTCTTCTGTCAGACTTCCTCTTTGTACCACAGTGGCTCTGGGGCCAGGCCTGCCTGCCCACTGGCCATCG
CCACCTTCCCCAGCTGCCTCCTACCAGCAGTTTCTCTGAAGATCTGTCAACAGGTTAAGTCAATCTGGGG
CTTCCACTGCCTGCATTCCAGTCCCCAGAGCTTGGTGGTCCCGAAACGGGAAGTACATATTGGGGCATGG
TGGCCTCCGTGAGCAAATGGTGTCTTGGGCAATCTGAGGCCAGGACAGATGTTGCCCCACCCACTGGAGA
TGGTGCTGAGGGAGGTGGGTGGGGCCTTCTGGGAAGGTGAGTGGAGAGGGGCACCTGCCCCCCGCCCTCC
CCATCCCCTACTCCCACTGCTCAGCGCGGGCCATTGCAAGGGTGCCACACAATGTCTTGTCCACCCTGGG
ACACTTCTGAGTATGAAGCGGGATGCTATTAAAAACTACATGGGGAAACAGGTGCAAACCCTGGAGATGG
ATTGTAAGAGCCAGTTTAAATCTGCACTCTGCTGCTCCTCCCCCACCCCCACCTTCCACTCCATACAATC
TGGGCCTGGTGGAGTCTTCGCTTCAGAGCCATTCGGCCAGGTGCGGGTGATGTTCCCATCTCCTGCTTGT
GGGCATGCCCTGGCTTTGTTTTTATACACATAGGCAAGGTGAGTCCTCTGTGGAATTGTGATTGAAGGAT
TTTAAAGCAGGGGAGGAGAGTAGGGGGCATCTCTGTACACTCTGGGGGTAAAACAGGGAAGGCAGTGCCT
GAGCATGGGGACAGGTGAGGTGGGGCTGGGCAGACCCCCTGTAGCGTTTAGCAGGATGGGGGCCCCAGGT
ACTGTGGAGAGCATAGTCCAGCCTGGGCATTTGTCTCCTAGCAGCCTACACTGGCTCTGCTGAGCTGGGC
CTGGGTGCTGAAAGCCAGGATTTGGGGCTAGGCGGGAAGATGTTCGCCCAATTGCTTGGGGGGTTGGGGG
GATGGAAAAGGGGAGCACCTCTAGGCTGCCTGGCAGCAGTGAGCCCTGGGCCTGTGGCTACAGCCAGGGA
ACCCCACCTGGACACATGGCCCTGCTTCTAAGCCCCCCAGTTAGGCCCAAAGGAATGGTCCACTGAGGGC
CTCCTGCTCTGCCTGGGCTGGGCCAGGGGCTTTGAGGAGAGGGTAAACATAGGCCCGGAGATGGGGCTGA
CACCTCGAGTGGCCAGAATATGCCCAAACCCCGGCTTCTCCCTTGTCCCTAGGCAGAGGGGGGTCCCTTC
TTTTGTTCCCTCTGGTCACCACAATGCTTGATGCCAGCTGCCATAGGAAGAGGGTGCTGGCTGGCCATGG
TGGCACACACCTGTCCTCCCAGCACTTTGCAGGGCTGAGGTGGAAGGACCGCTTAAGCCCAGGTGTTCAA
GGCTGCTGTGAGCTGTGTTCGAGCCACTACACTCCAGCCTGGGGACGGAGCAAAACTTTGCCTCAAAACA
AATTTTAAAAAGAAAGAAAGAAGGAAAGAGGGTATGTTTTTCACAATTCATGGGGGCCTGCATGGCAGGA
GTGGGGACAGGACACCTGCTGTTCCTGGAGTCGAAGGACAAGCCCACAGCCCAGATTCCGGTTCTCCCAA
CTCAGGAAGAGCATGCCCTGCCCTCTGGGGAGGCTGGCCTGGCCCCAGCCCTCAGCTGCTGACCTTGAGG
CAGAGACAACTTCTAAGAATTTGGCTGCCAGACCCCAGGCCTGGCTGCTGCTGTGTGGAGAGGGAGGCGG
CCCGCAGCAGAACAGCCACCGCACTTCCTCCTCAGCTTCCTCTGGTGCGGCCCTGCCCTCTCTTCTCTGG
ACCCTTTTACAACTGAACGCATCTGGGCTTCGTGGTTTCCTGTTTTCAGCGAAATTTACTCTGAGCTCCC
AGTTCCATCTTCATCCATGGCCACAGGCCCTGCCTACAACGCACTAGGGACGTCCCTCCCTGCTGCTGCT
GGGGAGGGGCAGGCTGCTGGAGCCGCCCTCTGAGTTGCCCGGGATGGTAGTGCCTCTGATGCCAGCCCTG
GTGGCTGTGGGCTGGGGTGCATGGGAGAGCTGGGTGCGAGAACATGGCGCCTCCAGGGGGCGGGAGGAGC
ACTAGGGGCTGGGGCAGGAGGCTCCTGGAGCGCTGGATTCGTGGCACAGTCTGAGGCCCTGAGAGGGAAA
TCCATGCTTTTAAGAACTAATTCATTGTTAGGAGATCAATCAGGAATTAGGGGCCATCTTACCTATCTCC
TGACATTCACAGTTTAATAGAGACTTCCTGCCTTTATTCCCTCCCAGGGAGAGGCTGAAGGAATGGAATT
GAAAGCACCATTTGGAGGGTTTTGCTGACACAGCGGGGACTGCTCAGCACTCCCTAAAAACACACCATGG
AGGCCACTGGTGACTGCTGGTGGGCAGGCTGGCCCTGCCTGGGGGAGTCCGTGGCGATGGGCGCTGGGGT
GGAGGTGCAGGAGCCCCAGGACCTGCTTTTCAAAAGACTTCTGCCTGACCAGAGCTCCCACTACATGCAG
TGGCCCAGGGCAGAGGGGCTGATACATGGCCTTTTTCAGGGGGTGCTCCTCGCGGGGTGGACTTGGGAGT
GTGCAGTGGGACAGGGGGCTGCAGGGGTCCTGCCACCACCGAGCACCAACTTGGCCCCTGGGGTCCTGCC
TCATGAATGAGGCCTTCCCCAGGGCTGGCCTGACTGTGCTGGGGGCTGGGTTAACGTTTTCTCAGGGAAC
CACAATGCACGAAAGAGGAACTGGGGTTGCTAACCAGGATGCTGGGAACAAAGGCCTCTTGAAGCCCAGC
CACAGCCCAGCTGAGCATGAGGCCCAGCCCATAGACGGCACAGGCCACCTGGCCCATTCCCTGGGCATTC
CCTGCTTTGCATTGCTGCTTCTCTTCACCCCATGGAGGCTATGTCACCCTAACTATCCTGGAATGTGTTG
AGAGGGATTCTGAATGATCAATATAGCTTGGTGAGACAGTGCCGAGATAGATAGCCATGTCTGCCTTGGG
CACGGGAGAGGGAAGTGGCAGCATGCATGCTGTTTCTTGGCCTTTTCTGTTAGAATACTTGGTGCTTTCC
AACACACTTTCACATGTGTTGTAACTTGTTTGATCCACCCCCTTCCCTGAAAATCCTGGGAGGTTTTATT
GCTGCCATTTAACACAGAGGGCAATAGAGGTTCTGAAAGGTCTGTGTCTTGTCAAAACAAGTAAACGGTG
GAACTACGACTAAA
>NM_001024736.1 Homo sapiens CD276 molecule (CD276), transcript
variant 1, mRNA
SEQ ID NO: 8
CCGGCCTCAGGGACGCACCGGAGCCGCCTTTCCGGGCCTCAGGCGGATTCTCCGGCGCGGCCCGCCCCGC
CCCTCGGACTCCCCGGGCCGCCCCCGGCCCCCATTCGGGCCGGGCCTCGCTGCGGCGGCGACTGAGCCAG
GCTGGGCCGCGTCCCTGAGTCCCAGAGTCGGCGCGGCGCGGCAGGGGCAGCCTTCCACCACGGGGAGCCC
AGCTGTCAGCCGCCTCACAGGAAGATGCTGCGTCGGCGGGGCAGCCCTGGCATGGGTGTGCATGTGGGTG
CAGCCCTGGGAGCACTGTGGTTCTGCCTCACAGGAGCCCTGGAGGTCCAGGTCCCTGAAGACCCAGTGGT
GGCACTGGTGGGCACCGATGCCACCCTGTGCTGCTCCTTCTCCCCTGAGCCTGGCTTCAGCCTGGCACAG
CTCAACCTCATCTGGCAGCTGACAGATACCAAACAGCTGGTGCACAGCTTTGCTGAGGGCCAGGACCAGG
GCAGCGCCTATGCCAACCGCACGGCCCTCTTCCCGGACCTGCTGGCACAGGGCAACGCATCCCTGAGGCT
GCAGCGCGTGCGTGTGGCGGACGAGGGCAGCTTCACCTGCTTCGTGAGCATCCGGGATTTCGGCAGCGCT
GCCGTCAGCCTGCAGGTGGCCGCTCCCTACTCGAAGCCCAGCATGACCCTGGAGCCCAACAAGGACCTGC
GGCCAGGGGACACGGTGACCATCACGTGCTCCAGCTACCAGGGCTACCCTGAGGCTGAGGTGTTCTGGCA
GGATGGGCAGGGTGTGCCCCTGACTGGCAACGTGACCACGTCGCAGATGGCCAACGAGCAGGGCTTGTTT
GATGTGCACAGCATCCTGCGGGTGGTGCTGGGTGCAAATGGCACCTACAGCTGCCTGGTGCGCAACCCCG
TGCTGCAGCAGGATGCGCACAGCTCTGTCACCATCACACCCCAGAGAAGCCCCACAGGAGCCGTGGAGGT
CCAGGTCCCTGAGGACCCGGTGGTGGCCCTAGTGGGCACCGATGCCACCCTGCGCTGCTCCTTCTCCCCC
GAGCCTGGCTTCAGCCTGGCACAGCTCAACCTCATCTGGCAGCTGACAGACACCAAACAGCTGGTGCACA
GTTTCACCGAAGGCCGGGACCAGGGCAGCGCCTATGCCAACCGCACGGCCCTCTTCCCGGACCTGCTGGC
ACAAGGCAATGCATCCCTGAGGCTGCAGCGCGTGCGTGTGGCGGACGAGGGCAGCTTCACCTGCTTCGTG
AGCATCCGGGATTTCGGCAGCGCTGCCGTCAGCCTGCAGGTGGCCGCTCCCTACTCGAAGCCCAGCATGA
CCCTGGAGCCCAACAAGGACCTGCGGCCAGGGGACACGGTGACCATCACGTGCTCCAGCTACCGGGGCTA
CCCTGAGGCTGAGGTGTTCTGGCAGGATGGGCAGGGTGTGCCCCTGACTGGCAACGTGACCACGTCGCAG
ATGGCCAACGAGCAGGGCTTGTTTGATGTGCACAGCGTCCTGCGGGTGGTGCTGGGTGCGAATGGCACCT
ACAGCTGCCTGGTGCGCAACCCCGTGCTGCAGCAGGATGCGCACGGCTCTGTCACCATCACAGGGCAGCC
TATGACATTCCCCCCAGAGGCCCTGTGGGTGACCGTGGGGCTGTCTGTCTGTCTCATTGCACTGCTGGTG
GCCCTGGCTTTCGTGTGCTGGAGAAAGATCAAACAGAGCTGTGAGGAGGAGAATGCAGGAGCTGAGGACC
AGGATGGGGAGGGAGAAGGCTCCAAGACAGCCCTGCAGCCTCTGAAACACTCTGACAGCAAAGAAGATGA
TGGACAAGAAATAGCCTGACCATGAGGACCAGGGAGCTGCTACCCCTCCCTACAGCTCCTACCCTCTGGC
TGCAATGGGGCTGCACTGTGAGCCCTGCCCCCAACAGATGCATCCTGCTCTGACAGGTGGGCTCCTTCTC
CAAAGGATGCGATACACAGACCACTGTGCAGCCTTATTTCTCCAATGGACATGATTCCCAAGTCATCCTG
CTGCCTTTTTTCTTATAGACACAATGAACAGACCACCCACAACCTTAGTTCTCTAAGTCATCCTGCCTGC
TGCCTTATTTCACAGTACATACATTTCTTAGGGACACAGTACACTGACCACATCACCACCCTCTTCTTCC
AGTGCTGCGTGGACCATCTGGCTGCCTTTTTTCTCCAAAAGATGCAATATTCAGACTGACTGACCCCCTG
CCTTATTTCACCAAAGACACGATGCATAGTCACCCCGGCCTTGTTTCTCCAATGGCCGTGATACACTAGT
GATCATGTTCAGCCCTGCTTCCACCTGCATAGAATCTTTTCTTCTCAGACAGGGACAGTGCGGCCTCAAC
ATCTCCTGGAGTCTAGAAGCTGTTTCCTTTCCCCTCCTTCCTCCTCTTGCTCTAGCCTTAATACTGGCCT
TTTCCCTCCCTGCCCCAAGTGAAGACAGGGCACTCTGCGCCCACCACATGCACAGCTGTGCATGGAGACC
TGCAGGTGCACGTGCTGGAACACGTGTGGTTCCCCCCTGGCCCAGCCTCCTCTGCAGTGCCCCTCTCCCC
TGCCCATCCTCCCCACGGAAGCATGTGCTGGTCACACTGGTTCTCCAGGGGTCTGTGATGGGGCCCCTGG
GGGTCAGCTTCTGTCCCTCTGCCTTCTCACCTCTTTGTTCCTTTCTTTTCATGTATCCATTCAGTTGATG
TTTATTGAGCAACTACAGATGTCAGCACTGTGTTAGGTGCTGGGGGCCCTGCGTGGGAAGATAAAGTTCC
TCCCTCAAGGACTCCCCATCCAGCTGGGAGACAGACAACTAACTACACTGCACCCTGCGGTTTGCAGGGG
GCTCCTGCCTGGCTCCCTGCTCCACACCTCCTCTGTGGCTCAAGGCTTCCTGGATACCTCACCCCCATCC
CACCCATAATTCTTACCCAGAGCATGGGGTTGGGGCGGAAACCTGGAGAGAGGGACATAGCCCCTCGCCA
CGGCTAGAGAATCTGGTGGTGTCCAAAATGTCTGTCCAGGTGTGGGCAGGTGGGCAGGCACCAAGGCCCT
CTGGACCTTTCATAGCAGCAGAAAAGGCAGAGCCTGGGGCAGGGCAGGGCCAGGAATGCTTTGGGGACAC
CGAGGGGACTGCCCCCCACCCCCACCATGGTGCTATTCTGGGGCTGGGGCAGTCTTTTCCTGGCTTGCCT
CTGGCCAGCTCCTGGCCTCTGGTAGAGTGAGACTTCAGACGTTCTGATGCCTTCCGGATGTCATCTCTCC
CTGCCCCAGGAATGGAAGATGTGAGGACTTCTAATTTAAATGTGGGACTCGGAGGGATTTTGTAAACTGG
GGGTATATTTTGGGGAATAATGTCTTTGTAAGCTTAAAAAAAAAAAAAAAAAAAAAAAA

As used herein, the term “tubulin” has its general meaning in the art and refers to a member of the tubulin protein superfamily of globular proteins, or one of the member proteins of that superfamily. The tubulin superfamily includes five distinct families, the alpha-, beta-, gamma-, delta-, and epsilon-tubulins and a sixth family (zeta-tubulin) which is present only in kinetoplastid protozoa. The most common members of the tubulin family are alpha-tubulin (α-tubulin) and beta-tubulin (β-tubulin), the proteins that make up microtubules. In particular, the term “tubulin” thus refers to α- and β-tubulins that polymerize into microtubules, a major component of the eukaryotic cytoskeleton. For reference, human alpha-tubulin is sequence SEQ ID NO:9 (Q71U36-1) and human beta-tubulin is sequence SEQ ID NO: 10 (P07437. Q71U36-1 human alpha tubulin)

SEQ ID NO: 9
MRECISIHVGQAGVQIGNACWELYCLEHGIQPDGQMPSDKTIGGGDDSFN
TFFSETGAGKHVPRAVFVDLEPTVIDEVRTGTYRQLFHPEQLITGKEDAA
NNYARGHYTIGKEIIDLVLDRIRKLADQCTRLQGFLVFHSFGGGTGSGFT
SLLMERLSVDYGKKSKLEFSIYPAPQVSTAVVEPYNSILTTHTTLEHSDC
AFMVDNEAIYDICRRNLDIERPTYTNLNRLISQIVSSITASLRFDGALNV
DLTEFQTNLVPYPRIHFPLATYAPVISAEKAYHEQLSVADITNACFEPAN
QMVKCDPGHGKYMACCLLYRGDVVPKDVNAAIATIKTKRTIQFVDWCPTG
EKVGINYQPPTVVPGGDLAKVQRAVCMLSNTTAIAEAWARLDHKFDLMYA
KRAFVHWYVGEGMEEGEFSEAREDMAALEKDYEEVGVDSVEGEGEEEGEE
Y
SEQ ID NO: 10
MREIVHIQAGQCGNQIGAKFWEVISDEHGIDPTGTYHGDSDLQLDRISVY
YNEATGGKYVPRAILVDLEPGTMDSVRSGPFGQIFRPDNFVFGQSGAGNN
WAKGHYTEGAELVDSVLDVVRKEAESCDCLQGFQLTHSLGGGTGSGMGTL
LISKIREEYPDRIMNTFSVVPSPKVSDTVVEPYNATLSVHQLVENTDETY
CIDNEALYDICFRTLRLTTPTYGDLNHLVSGTMECVTTCLRFPGQLNADL
RKLAVNMVPFPRLHFFMPGFAPLTSRGSQQYRALTVPDLTQQVFDAKNMM
AACDPRHGRYLTVAAVFRGRMSMKEVDEQMLNVQNKNSSYFVEWIPNNVK
TAVCDIPPRGLKMAVTFIGNSTAIQELFKRISEQFTAMFRRKAFLHWYTG
EGMDEMEFTEAESNMNDLVSEYQQYQDATAEEEEDFGEEAEEEA

As used herein, the term “polymerized-tubulin moiety” refers to a polymeric form of tubulin. As used herein, the term ‘polymerized tubulin” refers exclusively to the assembly of monomeric tubulin, or alternatively of the assembly of heterodimers of tubulin, in a regular fashion and with a distinct polarity. Tubular polymers of tubulin can grow as long as 50 micrometres, with an average length of 25 μm, and are highly dynamic. The outer diameter of a microtubule is generally of about 24-25 nm while the inner diameter is of about 12 nm. They are found in eukaryotic cells and are formed by the polymerization of a dimer of two globular proteins, α-tubulin and β-tubulin. Thus, the expression “polymerized tubulin” encompasses microtubules. The term “microtubule” has its general meaning in the art and represents a particular rearrangement of “polymerized tubulin”, which occurs physiologically in eukaryotic cells, and which forms with additional partners the “microtubule cytoskeleton”. The physiological assembly of microtubules is generally described as comprising a first step of regulated assembly of alpha-tubulin and beta-tubulin heterodimers, which together form a polarized protofilament. Then, protofilaments are believed to assemble, as a cylinder, into the so-called microtubule. Thus, microtubules are generally described as polymers of dimers of α- and β-tubulin, which are composed of 13 protofilaments assembled around a hollow core. However, it shall be noted that so-called microtubules with a different number of protofilaments have also been described in the Art, such as microtubules with 14 or 15 protofilaments.

Methods for determining the ability of a test compound to inhibit the binding of 2 partners are well known in the art and typically include surface plasmon resonance biosensors (Biacore®), saturation binding analysis with a labeled compound (for example, Scatchard and Lindmo analysis), differential UV spectrophotometer, fluorescence assay, Fluorometric Imaging Plate Reader (FLIPR®) system, Fluorescence resonance energy transfer, or Bioluminescence resonance energy transfer. Typically the methods involve the immobilization of one of the partner to a solid surface. Then the second partner is incubated with the previously immobilized first partner, in the presence or absence of the test compound. Then the binding including the binding level, or the absence of binding between said partners is then detected by any appropriate method. The term “solid surface” refers to a material having a rigid or semi-rigid surface. Such materials will preferably take the form of small beads, pellets, disks, chips, or wafers, although other forms may be used. The supports are generally made of conventional materials, e.g., plastic polymers, cellulose, glass, ceramic, stainless steel alloy, and the like. In some embodiments the solid support is a bead which is optionally labelled with one or more spectrally distinct fluorescent dyes, a number of methods of making and using sets of distinguishable beads have been described in the literature. These include beads distinguishable by size, wherein each size bead, beads with two or more fluorescent dyes at varying concentrations, wherein the beads are identified by the levels of fluorescence dyes, and beads distinguishably labelled with two different dyes, wherein the beads are identified by separately measuring the fluorescence intensity of each of the dyes. The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In some embodiments, the solid support is a magnetic bead that can be used use in magnetic separation. Typically, the magnetic bead is preferably made of a magnetic material selected from the group consisting of metals (e.g. ferrum, cobalt and nickel), an alloy thereof and an oxide thereof. In some embodiments, the partner is immobilized onto the support by any conventional method well known in the art. For instance, the partner that is directly or indirectly attached to the solid support is biotinylated and attached to the support via streptavidin, avidin or neutravidin. It thus contemplated that modified forms of avidin or streptavidin are employed to bind or capture the biotinylated partner. A number of modified forms of avidin or streptavidin that bind biotin specifically are known. Such modified forms of avidin or streptavidin include, e.g., physically modified forms (Kohanski, R. A. and Lane, M. D. (1990) Methods Enzymol. 194-200), chemically modified forms such as nitro-derivatives (Morag, E., et al., Anal. Biochem. 243 (1996) 257-263) and genetically modified forms of avidin or streptavidin (Sano, T., and Cantor, C. R., Proc. Natl. Acad. Sci. USA 92 (1995) 3180-3184).

In some embodiments, the mRNA sequence encoding for the immune checkpoint protein is biotinylated and immobilized in beads calibrated in size and coated with streptavidin. Then the beads are incubated with a cell lysate which brings the tubulin element and any other molecule that could favor e.g. the polymerization of tubulin and the binding of tubulin to the immobilized mRNA sequence, such as RNA binding proteins. The cell lysate is typically prepared from any cell such as a cell line (e.g. HELA). The incubation is performed at a temperature and for a time sufficient for allowing the binding of tubulin to the mRNA sequence. For instance, the incubation is performed at about 37° C. and for about 10 min. In one embodiment, an amount of the test compound is contacted with the immobilized RNA sequence before the incubation with the cell lysate so that the assay will allow the identification of compounds that bind to the RNA sequence and that inhibit the binding of tubulin to the RNA sequence. Alternatively, the amount of test compound is contacted with the cell lysate before the incubation with the immobilized RNA sequence the assay will allow the identification of compounds that bind to tubulin and that inhibit the binding of tubulin to the RNA sequence. The binding is typically revealed with an antibody having specificity for tubulin and which is conjugated to a detectable label. Detectable labels include fluorochromes and are known to those of skill in the art, and can be selected from, fluorescein isothiocyanate (FITC), and QFITC Q(RITC); 2′,7′-difluorofluorescein (OREGON GREEN®); and the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896. The fluorescence is then quantified and finally compared to the fluorescence quantified in the absence of the test compound. A decreased in fluorescence indicates that the test compound is an inhibitor of the binding to the RNA sequence to tubulin and then reveals that the test compound is a putative immune checkpoint inhibitor. Illustratively, the efficiency of the test compound may be assessed by determining for which amount of the test compound the binding is inhibited. Accordingly various concentration of the test compound may be prepared and then evaluated in the assay. Further illustratively the quantified fluorescence is compared to the fluorescence quantified with at least one compound already identified as an inhibitor of the binding. The assay may thus typically involve multi-well plates. Performing a screen on many thousands of test compounds indeed requires parallel handling and processing of many test compounds and assay component reagents. Standard high throughput screens (“HTS”) use mixtures of test compounds and biological reagents along with some indicator compound loaded into wells in standard microtiter plates with 96, 384, 1034 wells. The fluorescence is typically quantified with a fluorescence reader device, which typically uses a CCD camera to image the whole area of the multi-well plate. The image is analyzed to calculate the total fluorescence per well. Using robotics, data processing and control software, liquid handling devices, and sensitive detectors, HTS allows a quickly conduct of millions of tests. Through this process one can rapidly identify the test compounds capable of being an immune checkpoint inhibitor.

The test compounds that have been positively selected at the end of the in vitro screening methods which have been described previously in the present specification may be subjected to further selection steps in view of further assaying its properties. For instance, the positively tested compound may be then assayed for its capacity of inhibiting mitosis in any proliferation assay. According to the invention test compounds that are capable of inhibiting the binding of the RNA sequence to tubulin without showing any anti-mitotic properties are preferably selected. Additionally the test compounds that have been positively selected with the general in vitro screening method as above described may be further selected for their ability to enhance the cytolytic activity of lymphoid cells. Said killing activity may be determined by any assay well known in the art. Typically said assay is an in vitro assay wherein CD8+ T cells are brought into contact with target cells (e.g. tumor target cells that are recognized and/or lysed by CD8+ T cells). For example, the positively selected test compound can further selected for the ability to increase specific lysis by CD8+ T cells by more than about 20%, preferably with at least about 30%, at least about 40%, at least about 50%, or more of the specific lysis obtained at the same effector: target cell ratio with CD8+ T cells or CD8+ T cell lines that are not contacted by the test compound. Examples of protocols for classical cytotoxicity assays are conventional. Finally, in vivo assays may be performed with the positively selected compounds in various animal model of cancer and infectious diseases. Such animal models are also well known in the art.

As used herein, the term “test compound” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably. More specifically, test compounds that can be selected with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test compounds are synthetic molecules, and others natural molecules. Test compounds are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs. The test compounds can be naturally occurring proteins or their fragments. Such test compounds can be obtained from a natural source, e.g., a cell or tissue lysate. Libraries of polypeptide agents can also be prepared, e.g., from a cDNA library commercially available or generated with routine methods. The test compounds can also be peptides, e.g., peptides of from about 5 to about 30 amino acids, with from about 5 to about 20 amino acids being preferred and from about 7 to about 15 being particularly preferred. The peptides can be digests of naturally occurring proteins, random peptides, or “biased” random peptides. The test compounds can also be “nucleic acids”. Nucleic acid test compounds can be naturally occurring nucleic acids, random nucleic acids, or “biased” random nucleic acids. For example, digests of prokaryotic or eukaryotic genomes can be similarly used as described above for proteins. In some embodiments, the test compounds are small molecules (e.g., molecules with a molecular weight of not more than about 1,000).

For example, the screening methods of the present invention led to the discovery that CI-980 and other microtubule-destabilizing drugs constitute novel immune checkpoint inhibitors (see EXAMPLE and FIG. 1). The immune checkpoint inhibitors identified by the screening methods of the present invention can be used for the treatment of cancer and infectious diseases.

Methods of Treatment:

A further object of the present invention relates to use of a microtubule inhibitor as an immune checkpoint inhibitor.

As used herein, the term “microtubule inhibitor” has its general meaning in the art and refers to any compound that inhibits structure, stability, or function of microtubules and include but are not limited to compounds that inhibit microtubule growth, modulate the dynamics of microtubules, induce the self-association of tubulin dimers into single-walled rings and spirals, promote microtubule polymerization and/or stabilization, induce the dissociation or depolymerization of microtubules, and/or inhibit microtubule-based transport. Accordingly, the microtubule inhibitors thus include microtubule-destabilizing drugs, microtubule-stabilizing drugs and microtubule-based transport inhibitors including kinesin inhibitors. Examples of microtubule-stabilizing drugs include paclitaxel, docetaxel, taxanes, epothilones such as epothilone B, epothilone D, sagopilone, ixabepilone, laulimalide, peloruside A, discodermolide, dictyostatin, cyclostreptin, eleutherobin, sarcodictyin. Examples of microtubule-based transport inhibitors including kinesin inhibitors such as rose bengal lactone, adociasulfates, monastrol, terpendole E, S-trityl-cysteine, dimethylenastron, gossypol, ispinesib, HR22C16, SB743921, AZ3146, GSK923295, MPI-0479605, ARQ621, 4SC-205, among others. In preferred embodiments, the microtubule inhibitor of the present invention agent is microtubule destabilizing agent. Microtubule destabilizing agents are well known in the art. Preferably, the microtubule inhibitor of the present invention is a microtubule-destabilizing agent, such as colchicine, CI-980, combretastatine A4, vincristine, vinblastine, vinorelbine (Navelbine®, Pierre Fabre), vindesine (Eldisine®), vinflunine (Javlor®, Pierre Fabre Medicament), ABT-751 (Abbott); verubulin hydrochloride (Azixa™, Myriad Pharmaceuticals), lexibulin hydrochloride (YM Biosciences Australia), denibulin (MediciNova/Angiogene), indibulin (Zybulin™, Ziopharm Oncology), halichondrin B, eribulin (Halaven®, Eisai Inc.), combrestatin A4 (Zybrestat™, Oxigene), combrestatin A1 (Oxi4053, Oxigene), AVE8062 (Sanofi-Aventis), auristatins, cryptophycins, dolastatins, podophyllotoxin, estramustin, or any pharmaceutically acceptable salt thereof. Other examples include those disclosed in WO2004/103994 A1, which is incorporated by reference herein. Specific example include BAL27862 (3-(4-{1-[2-(4-Amino-phenyl)-2-oxo-ethyl]-1H-benzoimidazol-2-yl}-furazan-3-ylamino)-propionitrile, which has the structure below:

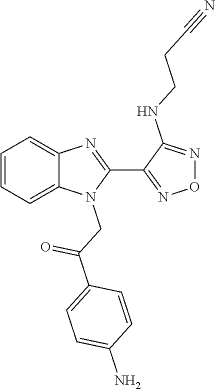 [SMILES: N#CCCNc1nonc1-c1nc2ccccc2n1CC(=O)c1ccc(N)cc1]

Further compounds exemplified in WO2004/103994 A1 as examples 50 and 79 respectively, and also specifically incorporated by cross-reference herein, have the structures and chemical names given below:

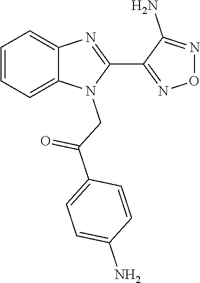 [SMILES: Nc1ccc(C(=O)Cn2c(-c3nonc3N)nc3ccccc32)cc1]

Chemical name: 2-[2-(4-Amino-furazan-3-yl)-benzoimidazol-1-yl]-1-(4-amino-phenyl)-ethanone; or herein as Compound B and

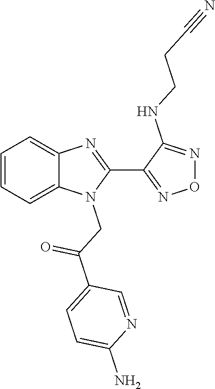 [SMILES: N#CCCNc1nonc1-c1nc2ccccc2n1CC(=O)c1ccc(N)nc1]

Chemical name: 3-(4-{1-[2-(6-Amino-pyridin-3-yl)-2-oxo-ethyl]-1H-benzoimidazol-2-yl}-furazan-3-ylamino)-propionitrile; or herein as Compound C.

In some embodiments, the microtubule inhibitor of the present invention is CI_980 ((S)-ethyl (5-amino-2-methyl-3-phenyl-1,2-dihydropyrido [3,4-b)]pyrazin-7-yl)carbamate), which has the formula of:

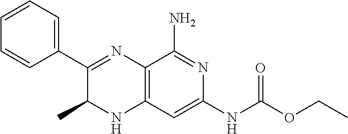 [SMILES: CCOC(=O)Nc1cc2c(c(N)n1)N=C(c1ccccc1)[C@H](C)N2]

Accordingly, the microtubule inhibitor of the present invention are particularly suitable for the treatment of cancer, by enhancing the intratumoral immune responses. As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangio sarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In particular, the inhibitor of the present invention is particularly suitable for the treatment of cancer characterized by high expression of immune checkpoint proteins (e.g. PD-1).

Accordingly, one further aspect of the present invention relates to a method of treating cancer in a patient in need thereof comprising i) determining the expression of at least one immune checkpoint protein selected from the group consisting of B7-H3, B7-H4, BTLA, CTLA-4, CD277, KIR, PD-1, LAG-3, TIM-3, TIGIT and VISTA, ii) comparing the determined expression level with a predetermined reference value and iii) administering to the patient a therapeutically effective amount of the microtubule inhibitor of the present invention when the determined expression level is higher than the predetermined reference value.

As used herein, the term “tumor tissue sample” means any tissue tumor sample derived from the patient. Said tissue sample is obtained for the purpose of the in vitro evaluation. In some embodiments, the tumor sample may result from the tumor resected from the patient. In some embodiments, the tumor sample may result from a biopsy performed in the primary tumour of the patient or performed in metastatic sample distant from the primary tumor of the patient. For example an endoscopical biopsy performed in the bowel of the patient suffering from the colorectal cancer. In some embodiments, the tumor tissue sample encompasses (i) a global primary tumor (as a whole), (ii) a tissue sample from the center of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and (vii) a distant metastasis. As used herein the “invasive margin” has its general meaning in the art and refers to the cellular environment surrounding the tumor. In some embodiments, the tumor tissue sample, irrespective of whether it is derived from the center of the tumor, from the invasive margin of the tumor, or from the closest lymph nodes, encompasses pieces or slices of tissue that have been removed from the tumor center of from the invasive margin surrounding the tumor, including following a surgical tumor resection or following the collection of a tissue sample for biopsy, for further quantification of one or several biological markers, notably through histology or immunohistochemistry methods, and through methods of gene or protein expression analysis, including genomic and proteomic analysis. The tumor tissue sample can be subjected to a variety of well-known post-collection preparative and storage techniques (e.g., fixation, storage, freezing, etc.) prior to determining the expression level of the gene of interest. Typically the tumor tissue sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (IHC) (using an IHC automate such as BenchMark® XT or Autostainer Dako, for obtaining stained slides). The tumour tissue sample can be used in microarrays, called as tissue microarrays (TMAs). TMA consist of paraffin blocks in which up to 1000 separate tissue cores are assembled in array fashion to allow multiplex histological analysis. This technology allows rapid visualization of molecular targets in tissue specimens at a time, either at the DNA, RNA or protein level. TMA technology is described in WO2004000992, U.S. Pat. No. 8,068,988, 011i et al 2001 Human Molecular Genetics, Tzankov et al 2005, Elsevier; Kononen et al 1198; Nature Medicine.

In some embodiments, the expression level is determined by determining the quantity of mRNA encoding for the immune checkpoint protein. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA). In some embodiments, the methods of the invention comprise the steps of providing total RNAs extracted from cumulus cells and subjecting the RNAs to amplification and hybridization to specific probes, more particularly by means of a quantitative or semi-quantitative RT-PCR. Alternatively, the expression level is determined by and ISH procedures (for example, fluorescence in situ hybridization (FISH), chromogenic in situ hybridization (CISH) and silver in situ hybridization (SISH)) or comparative genomic hybridization (CGH). In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques. Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. l. Pathol. 157:1467-1472, 2000 and U.S. Pat. No. 6,942,970. Additional detection methods are provided in U.S. Pat. No. 6,280,929. In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor tissue sample with a probe library, such that the presence of the target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

In some embodiments, the expression level is determined by determining the quantity of the immune checkpoint protein. Methods for quantifying protein of interest are well known in the art and typically involve immunohistochemistry. Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the immune checkpoint protein of interest, v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated with the binding partners having for the immune checkpoint protein of interest. After washing, the labeled antibodies that are bound to the immune checkpoint protein of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems. In some embodiments, the resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the immune checkpoint protein in the sample. Various automated sample processing, scanning and analysis systems suitable for use with IHC are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample.

Multiplex tissue analysis techniques might also be useful for quantifying several immune checkpoint proteins in the tumor tissue sample. Such techniques should permit at least five, or at least ten or more biomarkers to be measured from a single tumor tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, Md.). In some embodiments, the present methods utilize Multiplex Tissue Imprinting (MTI) technology for measuring biomarkers, wherein the method conserves precious biopsy tissue by allowing multiple biomarkers, in some cases at least six biomarkers. In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, Md.). That technique is described in U.S. Pat. No. 7,473,532. In some embodiments, the method of the present invention utilized the multiplex IHC technique developed by GE Global Research (Niskayuna, N.Y.). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected. In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image-processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the patient, ii) TMA samples are then stained with anti-antibodies having specificity of the immune checkpoint protein(s) of interest, iii) the TMA slide is further stained with an epithelial cell marker to assist in automated segmentation of tumor and stroma, iv) the TMA slide is then scanned using a multispectral imaging system, v) the scanned images are processed using an automated image analysis software (e.g.Perkin Elmer Technology) which allows the detection, quantification and segmentation of specific tissues through powerful pattern recognition algorithms. The machine-learning algorithm was typically previously trained to segment tumor from stroma and identify cells labelled.

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of expression level of the gene in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the gene in a group of reference, one can use algorithmic analysis for the statistic treatment of the measured expression levels of the gene(s) in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

The microtubule inhibitor of the present invention is also particularly suitable for the treatment of infectious diseases.

As used herein the term “infectious disease” includes any infection caused by viruses, bacteria, protozoa, molds or fungi. In some embodiments, the viral infection comprises infection by one or more viruses selected from the group consisting of Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae viruses. Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae viruses. In some embodiments, the viral infection comprises infection by one or more viruses selected from the group consisting of adenovirus, rhinovirus, hepatitis, immunodeficiency virus, polio, measles, Ebola, Coxsackie, Rhino, West Nile, small pox, encephalitis, yellow fever, Dengue fever, influenza (including human, avian, and swine), lassa, lymphocytic choriomeningitis, junin, machuppo, guanarito, hantavirus, Rift Valley Fever, La Crosse, California encephalitis, Crimean-Congo, Marburg, Japanese Encephalitis, Kyasanur Forest, Venezuelan equine encephalitis, Eastern equine encephalitis, Western equine encephalitis, severe acute respiratory syndrome (SARS), parainfluenza, respiratory syncytial, Punta Toro, Tacaribe, pachindae viruses, adenovirus, Dengue fever, influenza A and influenza B (including human, avian, and swine), junin, measles, parainfluenza, Pichinde, punta toro, respiratory syncytial, rhinovirus, Rift Valley Fever, severe acute respiratory syndrome (SARS), Tacaribe, Venezuelan equine encephalitis, West Nile and yellow fever viruses, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and Kyasanur forest disease. Bacterial infections that can be treated according to this invention include, but are not limited to, infections caused by the following: Staphylococcus; Streptococcus, including S. pyogenes; Enterococcus; Bacillus, including Bacillus anthracis, and Lactobacillus; Listeria; Corynebacterium diphtheriae; Gardnerella including G. vaginalis; Nocardiae; Streptomyces; Thermoactinomyces vulgaris; Treponema; Campylobacter, Pseudomonas including aeruginosa; Legionella; Neisseria including N. gonorrhoeae and N.meningitides; Flavobacterium including F. meningosepticum and F. odoraturn; Brucella; Bordetella including B. pertussis and B. bronchiseptica; Escherichia including E. coli, Klebsiella; Enterobacter, Serratia including S. marcescens and S. liquefaciens; Edwardsiella; Proteus including P. mirabilis and P. vulgaris; Streptobacillus; Rickettsiaceae including R. flickettsi, Chlamydia including C. psittaci and C. trachornatis; Mycobacteria including M. tuberculosis, M. intracellulare, M. fortuitum, M. leprae, M. avium, M. bovis, M. africanum, M. kansasii, M. gastri and Nocardiae. Protozoa infections that may be treated according to this invention include, but are not limited to, infections caused by plasmodia, leishmania, kokzidioa, and trypanosoma. A complete list of infectious diseases can be found on the website of the National Center for Infectious Disease (NCID) at the Center for Disease Control (CDC) (World Wide Web (www) at cdc.gov/ncidod/diseases/), which list is incorporated herein by reference. All of said diseases are candidates for treatment using the compositions according to the invention.

The formulation of the immune checkpoint inhibitor will depend upon factors such as the nature of the agent identified, the precise combination of symptoms, and the severity of the disease. Typically the immune checkpoint inhibitor is formulated for use with a pharmaceutically acceptable carrier or diluent. For example it may be formulated for intracranial, parenteral, intravenous, intramuscular, subcutaneous, transdermal or oral administration. A physician will be able to determine the required route of administration for each particular patient. The pharmaceutical carrier or diluent may be, for example, an isotonic solution. The dose of product may be determined according to various parameters, especially according to the substance used; the age, weight and condition of the patient to be treated; the route of administration; the severity of the disease, and the required regimen. A suitable dose may however be from 0.1 to 100 mg/kg body weight such as 1 to 40 mg/kg body weight. Again, a physician will be able to determine the required route of administration and dosage for any particular patient.

The invention will be further illustrated by the following figures and examples. However, these examples and figures should not be interpreted in any way as limiting the scope of the present invention.

### FIGURES

FIG. 1: Microtubule-targeting drugs inhibit PD-1 cell surface expression. Note that antimitotic drugs which do not target tubulin, like camptothecin (CPT), triptolide, mitomycin C, aphidicolin do not inhibit PD-1 expression. The non-binding-tubulin analogue of colchicine, beta-Lumi(−)-colchicine, does not affect PD-1 expression. Shown are means and standard deviations from more than ten independent experiments, using drugs at their respective optimum IC50 for proliferation.

FIG. 2: Microtubule-targeting drugs inhibit expression of PD-1 protein. CD3+T lymphocytes cell extracts, treated or not with CI-980, were analyzed by immunoblots. Figure shows a quantification from 3 independent experiments.

FIG. 3: Microtubule-targeting drugs increase PD-1 mRNA level. qRT-PCR analysis of mRNA extracted from activated CD3+ T lymphocytes treated or not with CI-980. Figure shows a quantification from 3 independent experiments.

FIG. 4: PD-1 protein is not associated with tubulin in T lymphocytes. CD3+T lymphocytes cell extract immunoprecipitated with anti-α-tubulin, anti-PD-1 or control isotype antibodies were tested for the presence of PD-1 protein by immunoblot (figure shows a representative result).

FIG. 5: PD-1 mRNA is associated with tubulin in T lymphocytes. CD3+T lymphocytes cell extract immunoprecipitated with anti-α-tubulin or control isotype antibodies were tested for presence of PD-1 mRNA by qRT-PCR (figure shows a quantification from 3 independent experiments).

FIG. 6: Tubulin binds to the ORF and 3′UTR of PD-1 mRNA. Whole-cell extracts prepared from CD3+ T lymphocytes were mixed with biotinylated full length, 5′UTR, ORF or 3′UTR regions of the PD-1 mRNA. Input and bound fractions were analyzed by immunoblotting with anti-α-tubulin, or antibodies to the specified RNA-binding proteins YB1 and HuR.

FIG. 7: Proteins interacting with PD-1 ORF mRNA include α-tubulin. Biotinylated PD-1 mRNA ORF coated on streptavidin sensor chips were eluted with whole cell extract, gently rinsed with buffer alone, prior to eluting with anti-α-tubulin, anti-actin or control isotype antibodies. Shown is a representative sensorgram demonstrating that tubulin was present among the cell lysate proteins interacting with PD-1 mRNA.

FIG. 8: Microtubule-targeting drugs inhibit expression of immune checkpoint markers LAG3, TIM3 and CTLA4 by activated T lymphocytes. CD3+ T lymphocyes were activated with anti-CD3/CD28 antibodies-coated beads plus IL-2, in the presence or absence of CI-980 (10 nM), and immune checkpoint expression were assessed by flow cytometry 3 days later.

FIG. 9: Microtubule-targeting drugs enhance cytolytic activity of immune cells. PBMC isolated from healthy donors were activated with anti-CD3/CD28 antibodies-coated beads plus IL-2, in the presence or absence of CI-980 (10 nM). After 3 days, PBMC were rinsed and co-incubated for 4 hours with the PD-L1⁺ cancer cell lines Karpas-299 or SU-DHL-1. Specific lysis of the target cell lines was then measured by staining of dying cells with propidium iodide (PI) and flow cytometry analysis.

### EXAMPLE

Material & Methods

Cell-Based Test of Inhibitors of Immune Checkpoint Expression by T Lymphocytes.

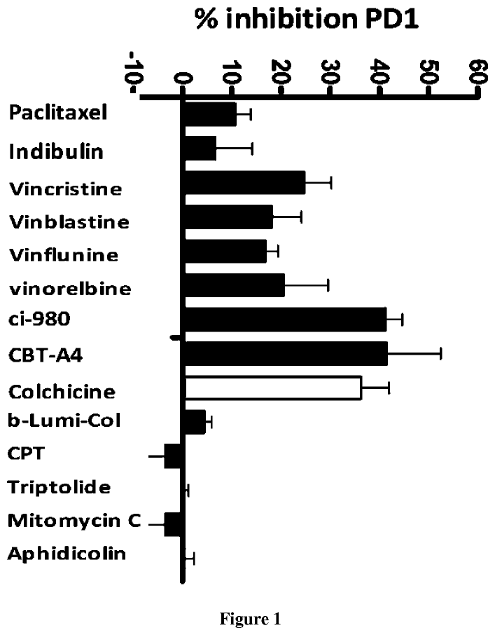

PBMC isolated from healthy donors were activated with CD3/CD28 antibodies-coated beads (ThermoFisher) and IL-2 (100 IU/ml) in the presence of the specified concentration of the tested drug. In the example (FIG. 1), these drugs include CI-980 (so-called NSC 613862 or mivobulin) (10 nM, or 1 nM and 0.1 nM as indicated in Figures), Combretastatin-A4 (CBT-A4), (100 nM), Colchicine (100 nM), Indibulin (100 nM), Vincristine (1 μM), Vinblastine (1 μM), Vinflunine (1 μM), Vinorelbine (1 μM), Paclitaxel (1 μM), 13-Lumi(−)-Colchicine (100 nM), Camptothecin (CPT) (1 μM), Triptolide (100 nM), Mitomycin C (1 μM), Aphidicolin (1 μM). After 3 days of in vitro cell culture in complete medium at 37° C. in a humidified atmosphere containing 5% CO2, cells were immunostained for CD3 (BioLegend, clone UCHT1), CD4 (BioLegend, clone RPA-T4), CD8 (BioLegend, clone RPA-T8), CD69 (BioLegend, clone FN50), CD279 (BD Biosciences, clone EH12.1), LAG3 (BioLegend, clone 11C3C65), TIM3 (BioLegend, clone F38-2E2) and CTLA4 (BioLegend, clone BNI3) expression and then analysed for immune checkpoint expression by flow cytometry and gating on the indicated subsets of T or NK cells.

Co-Immunoprecipitation.

α-tubulin was immunoprecipitated from activated CD3+T lymphocytes isolated from human PBMC of healthy donors using an anti-α-tubulin antibody (Sigma-Aldrich, clone B-5-1-2). Immunoprecipitation control with an anti-PD-1 antibody (ebioscience, clone J116) was also carried out. Immunoprecipitates were subjected to SDS-PAGE, and the co-immunoprecipitated PD-1 was assessed by Western Blot using an anti-PD-1 antibody (ThermoFisher, # PA5-20350).

RNA Immunoprecipitation (RIP).

Cell extract was realised from activated CD3+T lymphocytes isolated from human PBMC of healthy donors with polysomal lysis buffer (10 mM HEPES pH 7.0, 100 mM KCL, 5 mM MgCl2, 0.5% NP40, 1 mM DTT, 80 U RNase Inhibitor and protease Inhibitor cocktail (Roche)). Protein A/G PLUS agarose beads (Santa Cruz # sc-2003) (20 μl of slurry beads per μg of antibody) were coated with anti-α-tubulin or control anti-Ig antibody (18 μg per sample). The cell lysate (3 mg of protein) was diluted in the NT2 buffer (50 mM Tris-HCl pH 7.4, 150 mM NaCl, 1 mM MgCl2, 0.05% NP40) and incubated with antibody-coated beads, supplemented with 200 U RNase inhibitor per sample. 1/100e of the supernatant was kept as input for qRT-PCR analysis. After several washes, the beads were resuspended in Trizolreagent (Ambion) and RNA was extracted. Each RNA sample was treated with RQ1 RNase-free DNase (Promega) before proceeding to RT-PCR. Generation of cDNA was carried out with the RevertAid First Strand cDNA Synthesis Kit (ThermoFisher) according to the manufacturer's instruction. Real-time PCR assays were carried out with the ABI PRISM 7500 Real-Time PCR System (Applied Biosystems) using SYBR® Green JumpStart Taq Ready Mix™ (Sigma-Aldrich) with the primers PD1-99, 5′-CAGTTCCAAACCCTGGTGGT-3′ and PD1-100, 5′-GGCTCCTATTGTCCCTCGTG-3′ or GAPDH-107, 5′-CTCCTGTTCGACAGTCAGCC-3′ and GAPDH-108 5′-CTCCTGTTCGACAGTCAGCC-3′. GAPDH were used as reference gene. The amplification fold change was calculated with the ΔΔCT method.

RNA Affinity Chromatography.

In vitro synthesised biotinylated-mRNA was immobilised on streptavidin beads and incubated with whole cell extract from activated CD3+T lymphocytes. After extensive washing, mRNA-proteins complexes were resuspended in SDS-buffer, heated at 95° C. for 5 min, and subjected to SDS-PAGE. Proteins were transferred to nitrocellulose membranes, blotted with anti-α-tubulin, anti-YB1 or anti-HUR antibodies, and detected with HRP based enhanced chemiluminescence.

Surface Plasmon Resonance.

Binding between PD-1 mRNA and tubulin-containing protein complex was examined on a BIACORE T200 (GE Healthcare). In vitro synthesised biotinylated-mRNA was immobilized on streptavidin-coated sensors chip, and cellular extract was run through the sensor chip. Once the sensorgram reached the maximum amplitude, anti-α-tubulin, anti-actin or control anti-Ig antibody was injected.

Cytotoxicity Assay.

PBMC isolated from healthy donors, used as effector cells, were activated with CD3/CD28 beads and IL-2, in presence or absence of CI-980 (10 nM). Human cancer cell lines-expressing PD-L1 karpas-299 and SU-DHL-1 were used as targets cells. Prior to be mixed with the effector cells, target cells were labelled with CellTrace-Violet (ThermoFisher) in order to distinguish them from the effector cells. After 3 days, effector cells were rinsed and combined with target cells at a 2:1 effector-to-target ratio. Cells were incubated during 4 hours at 37° C. and specific lysis were analysed by flow cytometry. The percentage of lysis was determined as percent of cells positive for both propidium iodide (PI) and CellTrace-Violet versus cells positive for CellTrace-Violet.

Results

T lymphocytes activated by CD3/CD28 in presence of microtubule-targeting drugs show reduced expression of PD-1 and increased expression of CD69 (data not shown), suggesting these drugs inhibit the immune checkpoint PD-1 (data not shown). As illustrated for CD4+ and CD8+ T cells subtypes, this activity is observed on all T and NK cell subsets (data not shown). This activity on both PD-1 and CD69 is drug dose-dependent (data not shown).

Microtubule-targeting drugs can be categorised in two main classes, stabilizers or destabilizers. Most of the destabilizers are far more potent than stabilizers (as exemplified by paclitaxel) in cell surface PD-1 inhibition (FIG. 1). Since non-microtubule antimitotic drugs (such as camptothecin, triptolide, mitomycin C or aphidicolin) and the colchicine analog that does not bind tubulin (β-Lumi(−)-Colchicine) do not impact PD-1 expression (FIG. 1), the microtubule-destabilizing drugs (MDD) inhibit immune checkpoint expression.

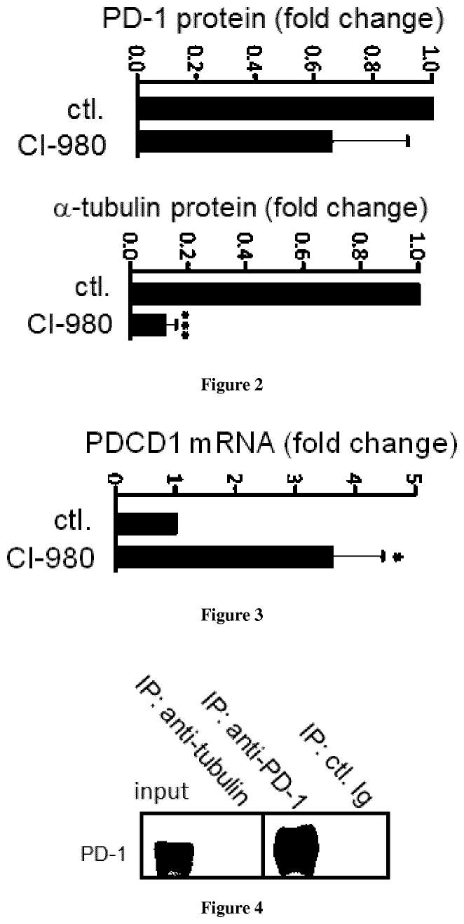

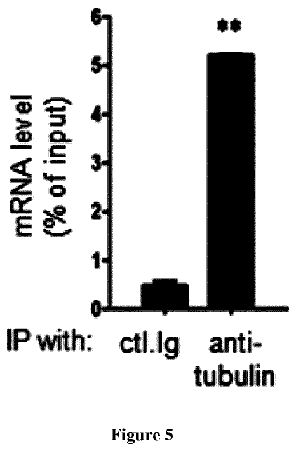

Western blot analysis of PD-1 and α-tubulin proteins confirms that MDD inhibit production of PD-1 protein and microtubule assembly (FIG. 2). However, qRT-PCR analysis demonstrates that MDD significantly increase the level of PD-1 mRNA from untreated cells (FIG. 3). Finally, co-immunoprecipitation and RNA immunoprecipitation experiments demonstrate that in untreated cell controls, tubulin does not interact with PD-1 protein (FIG. 4) but interacts with PD-1 mRNA (FIG. 5).

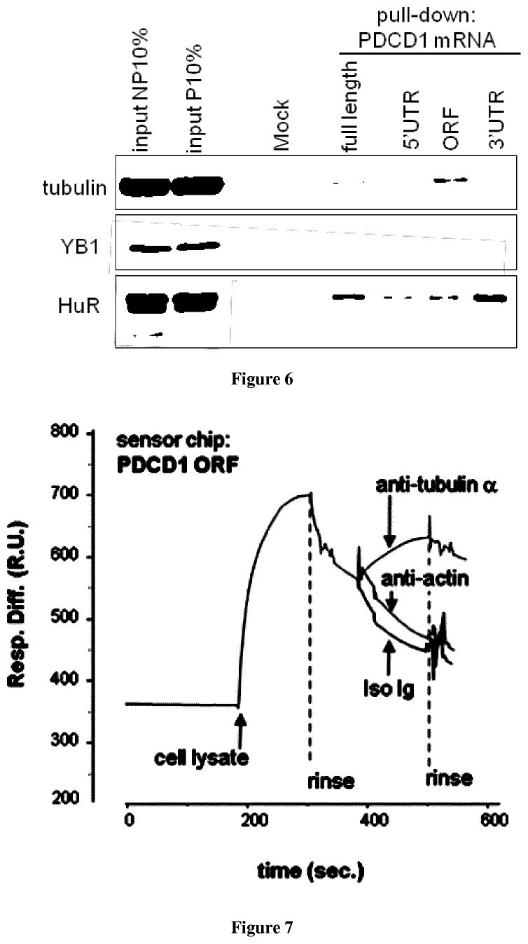

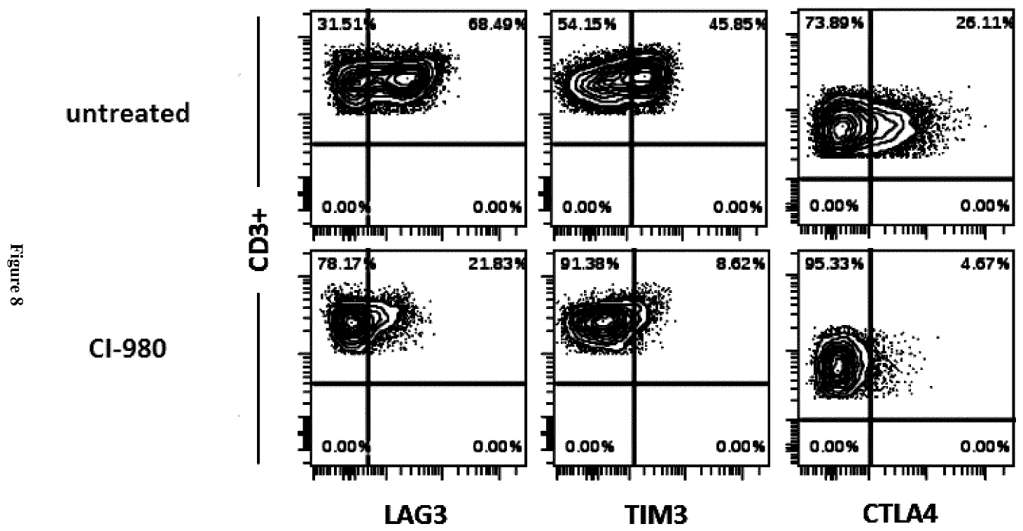

The region of PD-1 mRNA involved in tubulin association was identified by RNA pull-down experiments using different constructs of the PD-1 mRNA. The interaction of full-length PD-1 mRNA with α-tubulin was also observed with the ORF and, though to a lesser extent, the 3′UTR, but not by the 5′UTR (FIG. 6). The binding between PD-1 ORF mRNA and α-tubulin from cell extract was confirmed by surface plasmon resonance (FIG. 7).

So in normal activated T lymphocytes, intact microtubules interact with PD-1 mRNA to allow further translation and cell surface expression of the PD-1 immune checkpoint. Consequently, MDD abrogate these interactions and inhibit PD-1 expression.

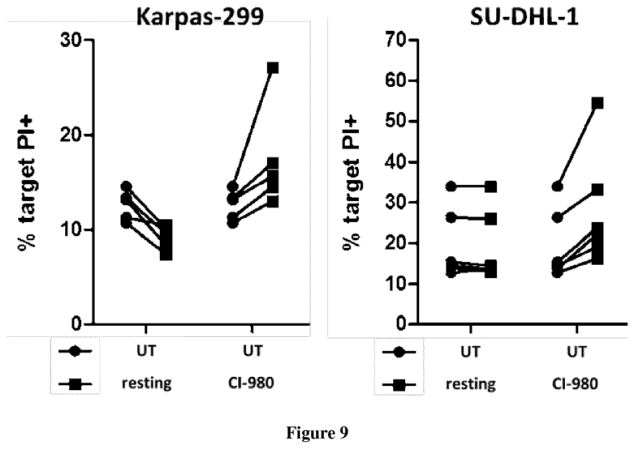

Activation of naïve T cells induces cell surface expression of PD-1 as well as several other inhibitory receptors such as CTLA4, LAG3 and TIM3. Treatment of the activated T lymphocytes with CI-980 or with other MDD also inhibits their expression of CTLA4, LAG3 and TIM3 (FIG. 8). Thus MDD and other inhibitors of RNA binding to microtubules constitute novel inhibitors of immune checkpoint expression. Consequently, these molecules enhance the cytolytic response of cytotoxic T and NK cells against target cells such as human cancer cells (FIG. 9).

### REFERENCES

Throughout this application, various references describe the state of the art to which this invention pertains. The disclosures of these references are hereby incorporated by reference into the present disclosure.

1. A method of screening an immune checkpoint inhibitor comprising
a) determining the ability of a test compound to inhibit binding of a mRNA sequence encoding an immune checkpoint protein to a polymerized-tubulin moiety, by
i) incubating the mRNA sequence and the polymerized-tubulin moiety together in the presence of the test compound; and
ii) detecting whether a complex forms between the mRNA sequence and the polymerized-tubulin moiety; and
b) positively selecting the test compound when the test compound inhibits the binding of the mRNA sequence.

2. The method of claim 1 wherein the mRNA sequence encodes for an immune checkpoint protein selected from the group consisting of PD-1, B7-H3, B7-H4, BTLA, CTLA-4, CD277, KIR, LAG-3, TIM-3, TIGIT and VISTA.

3. The method of claim 1 wherein the mRNA sequence corresponds to an open reading frame (ORF) region.

4. The method of claim 1 wherein the mRNA sequence corresponds to the transcription of a sequence selected from the group consisting of SEQ ID NO:1-8.

5. The method of claim 1, wherein the mRNA sequence encoding for the immune checkpoint protein or the polymerized-tubulin moiety is immobilized on a solid surface.

6. The method of claim 5 wherein the mRNA sequence encoding for the immune checkpoint protein is biotinylated and immobilized on beads calibrated in size and coated with streptavidin.

7. The method of claim 6 further comprising a step of incubating the beads with a cell lysate comprising one or more molecules that could affect microtubule dynamics and/or the interaction of tubulin with the mRNA sequence.

8. The method of claim 7 wherein the test compound is contacted with the immobilized RNA sequence before the step of incubating.

9. The method of claim 7 wherein the test compound is contacted with the cell lysate before the step of incubating.

10. The method of claim 8 wherein the binding is detected with an antibody having specificity for tubulin or microtubule-associated proteins, and wherein the antibody is conjugated to a detectable label.

11. The method of claim 1, further comprising a step of assaying the ability of the positively selected test compound to inhibit mitosis.

12-13. (canceled)

14. A method of treating cancer in a patient in need thereof comprising i) determining the expression level of at least one immune checkpoint protein selected from the group consisting of B7-H3, B7-H4, BTLA, CTLA-4, CD277, KIR, PD-1, LAG-3, TIM-3, TIGIT and VISTA, ii) comparing the expression level with a predetermined reference value and iii) administering to the patient a therapeutically effective amount of the microtubule inhibitor when the expression level is higher than the predetermined reference value.

15. The method of claim 7, wherein the one or more molecules are RNA binding proteins.
